(19) United States
(12) Patent Application Publication
(10) Pub. No.: US 2014/0336369 A1
(43) Pub. Date: Nov. 13, 2014

(54) PROCESS FOR PREPARING HEPARINOIDS AND INTERMEDIATES USEFUL IN THE SYNTHESIS THEREOF

(71) Applicant: Apicore US LLC, Somerset, NY (US)

(72) Inventors: Ravishanker Kovi, Monroe, NJ (US); Ashish Naik, Piscataway, NJ (US); Brijesh Patel, Piscataway, NJ (US); Muralikrishna Madala, Hyderabad (IN)

(73) Assignee: APICORE US LLC, Somerset, NY (US)

(21) Appl. No.: 14/338,927

(22) Filed: Jul. 23, 2014

Related U.S. Application Data
(63) Continuation of application No. 13/170,471, filed on Jun. 28, 2011, now abandoned.

(51) Int. Cl. C07H 11/00; C07H 3/02; C07H 3/04; C07H 1/00; C07H 1/06; C07H 19/01

(52) U.S. Cl. CPC C07H 11/00; C07H 1/06; C07H 19/01; C07H 3/04; C07H 1/00; C07H 3/02
USPC 536/17.2; 536/21; 536/18.2; 536/55.3; 536/55.1; 536/55

(57) ABSTRACT

Processes are disclosed for the synthesis of the Factor Xa anticoagulant fondaparinux and related compounds. Protected pentasaccharide intermediates and efficient and scalable processes for the industrial scale production of fondaparinux sodium by conversion of the protected pentasaccharide intermediates via a sequence of deprotection and sulfonation reactions are provided.

### CROSS-REFERENCE TO RELATED APPLICATIONS

This application is a continuation of U.S. patent application Ser. No. 13/170,471 filed Jun. 28, 2011, the entirety of which is incorporated herein by reference.

### FIELD OF THE INVENTION

The presently disclosed subject matter relates to processes for the synthesis of the Factor Xa anticoagulant fondaparinux, and related compounds. The subject matter also relates to protected pentasaccharide intermediates and to an efficient and scalable process for the industrial scale production of fondaparinux sodium by conversion of the protected pentasaccharide intermediates via a sequence of deprotection and sulfonation reactions.

### BACKGROUND

Vascular thrombosis is a cardiovascular disease indicated by the partial or total occlusion of a blood vessel by a clot containing blood cells and fibrin. In arteries, it results predominantly from platelet activation and leads to heart attack, angina or stroke, whereas venous thrombosis results in inflammation and pulmonary emboli. The coagulation of blood is the result of a cascade of events employing various enzymes collectively known as activated blood coagulation factors. Heparin, a powerful anticoagulant, has been used since the late 1930's in the treatment of thrombosis. In its original implementation, tolerance problems were noted and so reduced dosage was suggested to reduce bleeding and improve efficacy. In the early 1970's, clinical trials did indeed indicate acceptable tolerance was obtainable whilst still preserving antithrombotic activity. Unfractionated heparin (UFH) is primarily used as an anticoagulant for both therapeutic and surgical indications, and is usually derived from either bovine lung or porcine mucosa. Amongst the modern uses of unfractionated heparin include management of unstable angina, as an adjunct to chemotherapy and anti-inflammatory treatment, and as a modulation agent for growth factors and treatment of hemodynamic disorders. In the late 1980's, the development of low molecular weight heparins (LMWHs) led to improvements in antithrombotic therapy. LMWHs are derived from UFH by such processes as chemical degradation, enzymatic depolymerization and y-radiation cleavage. This class of heparins has recently been used for treatment of trauma related thrombosis. Of particular interest is that the relative effects of LMWHs on platelets are minimal compared to heparin, providing an immediate advantage when treating platelet-compromised patients.

The degree of depolymerization of UFH can be controlled to obtain LMWHs of different lengths. Dosage requirements for the treatment of deep vein thrombosis (DVT) are significantly reduced when employing LMWH as opposed to UFH, although in general the efficacy of both therapeutics seems to be comparable. In addition, LMWH can be effective as an alternative therapeutic for patients who have developed sensitivity to UFH. Unfortunately, there has recently been a great deal of concern in the use of LMWH due to the perceived potential for cross-species viral contamination as a result of the animal source of the parent UFH.

One way of avoiding the possibility of cross-species contamination, is to prepare heparins by chemical synthesis. This method would also provide the opportunity to develop second generation heparins or heparinoids, which can be tailored to target particular biological events in the blood coagulation cascade. An investigation to determine the critical structural motif required for an important binding event in a coagulation cascade involving heparin, dates back to the 1970's. Some structural features of heparin were defined, but the binding domains of interest remained essentially undefined. Research conducted by Lindahl and co-workers (Lindahl, et al., Proc. Natl. Acad. Sci. USA, 1980, Vol. 77, No. 11, 6551-6555; Reisenfeld, et al., J. Bioi. Chem., 1981, Vol. 256, No. 5, 2389-2394) and separately by Choay and co-workers (Choay, et al., Annals N.Y. Academy of Sciences, 1981, 370, 644-649) eventually led to the determination that a pentasaccharide sequence constituted the critical binding domain for the pro-anticoagulant cofactor antithrombin Ill (AT-Ill). After determination of the critical heparin sugar sequence, complete chemical syntheses were embarked upon to further prove the theories. Complete syntheses of the pentasaccharide binding domain were completed at similar times by Sinay and co-workers and by Van Boeckel and co-workers (Sinay, et al., Carbohydrate Research, 132, (1984),C5-C9). Significant difficulties were encountered during both these reported syntheses. The synthesis by Van Boeckel and co-workers provided a method on a reasonable scale (156 mg of final product) and with improved yields compared to the Sinay synthesis, but still only provided an overall yield of 0.22%, (compared with 0.053% for the Sinay synthesis).

Fondaparinux sodium, or methyl O-2-deoxy-6-O-sulfo-2-(sulfoamino)-α-D-glucopyranosyl-(1→4)-O-β-D-glucopyranuronosyl-(1→4)-O-2-deoxy-3,6-di-O-sulfo-2-(sulfoamino)-α-D-glucopyranosyl-(1→4)-O-2-O-sulfo-α-L-idopyranuronosyl-(1→4)-2-deoxy-6-O-sulfo-2-(sulfoamino)-α-D-glucopyranoside, decasodium salt, has the following structural formula:

(1)

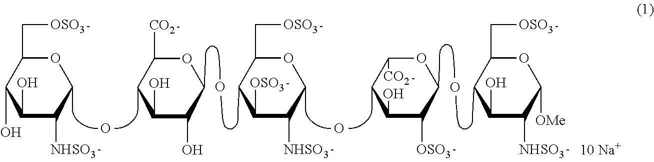

Fondaparinux sodium is a chemically synthesized methoxy derivative of the natural pentasaccharide sequence, which is the active site of heparin that mediates the interaction with antithrombin (Casu et al., J. Biochem., 197, 59, 1981). It has a challenging pattern of O- and N-sulfates, specific glycosidic stereochemistry, and repeating units of glucosamine and monic acids (Petitou et al., Progress in the Chemistry of Organic Natural Product, 60, 144-209, 1992). It is obtained according to the process described in EP 084,999 and U.S. Pat. No. 4,818,816.

Fondaparinux sodium is derived from a chemical synthesis having more than 50 steps. This process makes it possible to obtain crude fondaparinux sodium, which is a mixture consisting of fondaparinux sodium and other related oligosaccharides. The fondaparinux sodium content of this mixture, evaluated by anion exchange high performance liquid chromatography (HPLC), is approximately 70%. Several steps of purification by column chromatography and by precipitation are necessary in order to obtain fondaparinux sodium having better purity, however, even with these several purification steps the purity still does not exceed 96.0%. Furthermore, the large number of steps required for synthesis, involving the aforementioned column chromatography purification and long reaction times, makes it very difficult to standardize industrial batches.

Given the complexity of the structure of fondaparinux sodium and its synthesis intermediates, many impurities can form in the course of the synthesis. In addition, the slightest variation in the operating conditions results in batches of crude fondaparinux sodium being obtained which contain related but undesirable products in considerable amounts. These related products, which do not have anti-Xa activity or which have very slight activity, have a chemical structure and physicochemical characteristics which are very similar to fondaparinux sodium, and cannot be eliminated satisfactorily by the purification methods indicated above. Moreover, it has been observed that some of these products are readily degradable when they are subjected to sterilization by methods such as autoclaving, and thus produce additional impurities.

Fondaparinux sodium, the active principle in a pharmaceutical specialty product, must satisfy certain quality criteria and standards and must in particular be as highly pure as possible. As a result, industrial batches which contain related products in considerable amounts cannot be used for preparing pharmaceutical specialty products. Thus, it is important to have highly pure fondaparinux sodium compositions, and in particular industrial amounts of such compositions, and also a process for obtaining them.

Sugar oligomers or oligosaccharides such as fondaparinux are assembled using coupling reactions, also known as glycosylation reactions, to “link” sugar monomers together. The difficulty of this linking step arises because of the required stereochemical relationship between the D-sugar and the C-sugar, as shown below:

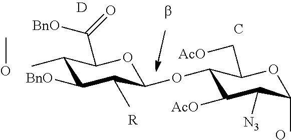

The stereo chemical arrangement illustrated above is described as having a configuration at the anomeric carbon of the D-sugar (denoted by the arrow). The linkage between the D and C units in fondaparinux has this specific stereochemistry. There are, however, competing β and α-glycosylation reactions.

The difficulties of the glycosylation reaction in the synthesis of fondaparinux are well known. In 1991 Sanofi reported a preparation of a disaccharide intermediate in 51% yield having a 12/1 ratio of β/α stereochemistry at the anomeric position (Duchaussoy et al., Bioorg. & Med. Chern. Lett., 1(2), 99-102, 1991). In Sinay e t al., Carbohydrate Research, 132, C5-C9, 1984, yields on the order of 50% with coupling times on the order of 6 days are reported. U.S. Pat. No. 4,818,816 (see e.g., column 31, lines 50-56) discloses a 50% yield for the glycosylation.

U.S. Pat. No. 7,541,445 is even less specific as to the details of the synthesis of this late-stage fondaparinux synthetic intermediate. The '445 patent discloses several strategies for the assembly of the pentasaccharide (1+4, 3+2 or 2+3) using a 2-acylated D-sugar (specifically 2-allyloxycarbonyl) for the glycosylation coupling reactions. However, the strategy involves late-stage pentasaccharides that all incorporate a 2-benzylated D sugar. The transformation of acyl to benzyl is performed either under acidic or basic conditions. Furthermore, these transformations, using benzyl bromide or benzyl trichloroacetimidate, typically result in extensive decomposition and the procedure suffers from poor yields. Thus, such transformations (at a disaccharide, trisaccharide, an d pentasaccharide level) are typically not acceptable for industrial scale production.

Examples of fully protected pentasaccharide are described in Duchaussoy et al., Bioorg. Med. Chern. Lett., 1 (2), 99-102, 1991; Petitou et al., Carbohydr. Res., 167, 67-75, 1987; Sinay et al., Carbohydr. Res., 132, C5-C9, 1984; Petitou et al., Carbohydr. Res., 1147, 221-236, 1986; Lei et al., Bioorg. Med. Chern., 6, 1337-1346, 1998; Ichikawa et al., Tet. Lett., 27(5), 611-614, 1986; Kovensky et al., Bioorg. Med. Chern., 1999, 7, 1567-1580, 1999. These fully protected pentasaccharides may be converted to the O- and N-sulfated pentasaccharides using the four steps (described earlier) of: a) saponification with LiOH/H₂O₂/NaOH, b) O-sulfation by an Et₃N—S0₃ complex; c) de-benzylation and azide reduction via H₂/Pd hydrogenation; and d)N-sulfation with a pyridine-S0₃ complex.

Even though many diverse analogs of the fully protected pentasaccharide have been prepared, none use any protective group at the 2-position of the D unit other than a benzyl group. Furthermore, none of the fully protected pentasaccharide analogs offer a practical, scalable and economical method for re-introduction of the benzyl moiety at the 2-position of the D unit after removal of any participating group that promotes glycosylation.

Furthermore, the coupling of benzyl protected sugars proves to be a sluggish, low yielding and problematic process, typically resulting in substantial decomposition of the pentasaccharide (prepared over 50 synthetic steps), thus making it unsuitable for a large (i.e., kilogram or more) scale production process.

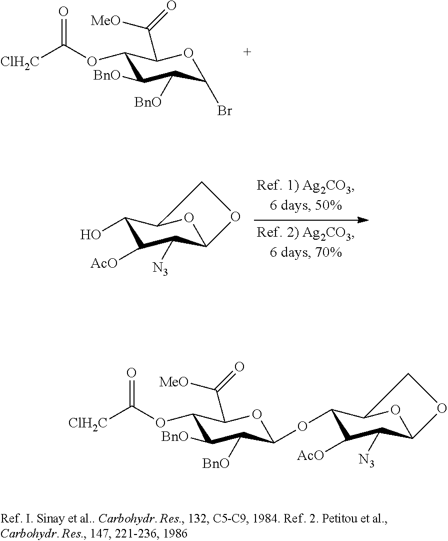

It has been a general strategy for carbohydrate chemists to use a base-labile ester-protecting group at the 2-position of the D unit to build an efficient and stereoselective glycosidic linkage. To construct the linkage carbohydrate chemists have previously employed acetate and benzoate ester groups, as described, for example, in the review by Poletti et al., Eur. J. Chern., 2999-3024, 2003.

The ester group at the 2-position of D needs to be differentiated from the acetate and benzoates at other positions in the pentasaccharide. These ester groups are hydrolyzed and sulfated later in the process and, unlike these ester groups, the 2-hydroxyl group of the D unit needs to remain as the hydroxyl group in the final product, fondaparinux sodium.

Some of the current ester choices for the synthetic chemists in the field include methyl chloro acetyl (MCA) and chloro methyl acetate (CMA). The mild procedures for the selective removal of theses groups in the presence of acetates and benzoates make them ideal candidates. However, MCA/CMA groups have been shown to produce unwanted and serious side products during glycosylation and therefore have not been favored in the synthesis of fondaparinux sodium and its analogs. For by-product formation observed in acetate derivatives see Seeberger et al., J. Org. Chem., 2004, 69, 4081-93. Similar by-product formation is also observed using chloroacetate derivatives. See Orgueira et al., Eur. J. Chern., 9(1), 140-169, 2003.

Therefore, as will be appreciated, there are several limitations and drawbacks in current processes used for the synthesis of fondaparinux sodium. Thus, there is a need in the art for new synthetic procedures that produce fondaparinux and related compounds efficiently, in high yield and with highly stereoselective purity, and which employ less expensive reagents and fewer hazardous materials.

### SUMMARY OF THE INVENTION

The processes presently disclosed address the limitations and drawbacks known in the art and provide a unique, reliable, efficient and scalable synthesis of compounds such as fondaparinux sodium. The present inventors have surprisingly found that in the synthesis of fondaparinux, the use of unique and improved reaction conditions and purification techniques allows for a highly efficient glycosylation reaction, thereby providing late-stage intermediates or oligosaccharides (and fondaparinux-related oligomers) in high yield and in high β/α ratios. In particular, glycosylation between two disaccharide units and tetrasaccharide and monosaccharide units can occur with high coupling yields (>65%) of the isomer, rapidly (for example, in an hour reaction time), and with no detectable α-isomer upon column chromatography purification. The new purification techniques permit elimination of column purification steps which are not suited to commercial production processes. The improved reaction conditions disclosed herein eliminate the lengthy and costly processes currently employed for the production of fondaparinux sodium and related intermediates, resulting in smooth and feasible processes which are acceptable for industrial scale production. In accordance with one aspect a first step involves acetolysis of chloro acetyl disaccharide sugar (CADS) carried out in the presence of acetic anhydride and trifluoroacetic acid (TFA) at ambient temperature. The resultant product residue, crystallized from ether instead of column chromatography, gives product in high yield and high purity.

A critical step of the disclosed processes which impacts all steps of the process is the bromination of acetylated CADS sugar, carried out in a mixture of moisture-free halogenated solvents such as methylene chloride, ethylene chloride and chloroform and ethyl acetate or butyl acetate in the presence of titanium bromide under argon atmosphere at reflux for 6 hrs. After work up, the residue is crystallized from a polar solvent such as methanol, ethanol, isopropanol, etc. instead of column chromatography, resulting in product in high yield and high purity.

Using the methods disclosed herein, far less solvent quantities are required than are used in prior art processes. Moreover, selectively purifying compounds at critical steps during the process surprisingly results in high yields and produces a final fondaparinux sodium product having a purity greater than 99.8% by HPLC, which is greater than that achievable using any prior art process. For example, in accordance with one aspect, distilling off the solvent dimethylformamide during preparation of the O-sulfonated pentasaccharide (L) surprisingly increased the yield of the final product by about 50%.

These and other aspects of the invention will be apparent to those skilled in the art.

### DETAILED DESCRIPTION OF THE INVENTION

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The following examples are merely illustrative of the present invention and should not be construed as limiting the scope of the invention in any way as many variations and equivalents that are encompassed by the present invention will become apparent to those skilled in the art upon reading the present disclosure.

### Examples

In the synthesis of Fondaparinux sodium, the monomers XII, XVIII, XXVII, XXXVIII, XXXXI and dimers XIX, XX, XL described herein may be made either by processes described in the art or, by a process as described herein. The XII and XVIII monomers may then linked to form a disaccharide XX, XXXIX and XXVII monomers may then linked to form a disaccharide XL, XLIII and XX dimers may then linked to form a tetrasaccharide, XLVII tetramer and XLV monomer may be linked to form a pentasaccharide (XLVIII) pentamer. The XLVIII pentamer is an intermediate that may be converted through a series of reactions to fondaparinux sodium. This strategy described herein provides an efficient method for multi-kilogram preparation of fondaparinux in high yields and highly stereoselective purity.

Synthetic Procedures

The following abbreviations are used herein: Ac is acetyl; MS is molecular sieve; DMF is dimethyl formamide; Bn is benzyl; MDC is dichloromethane; THF is tetrahydrofuran; TFA is trifluoro acetic acid; MeOH is methanol; RT is room temperature; Ac₂O is acetic anhydride; HBr is hydrogen bromide; EtOAc is ethyl acetate; Cbz is benzyloxycarbonyl; CADS is chloro acetyl disaccharide; HDS is hydroxy disaccharide; NMP is N-methylpyrrolidone.

### Methyl 3-O-benzyl-4-O-monochloro acetyl-β-L-idopyranuronate

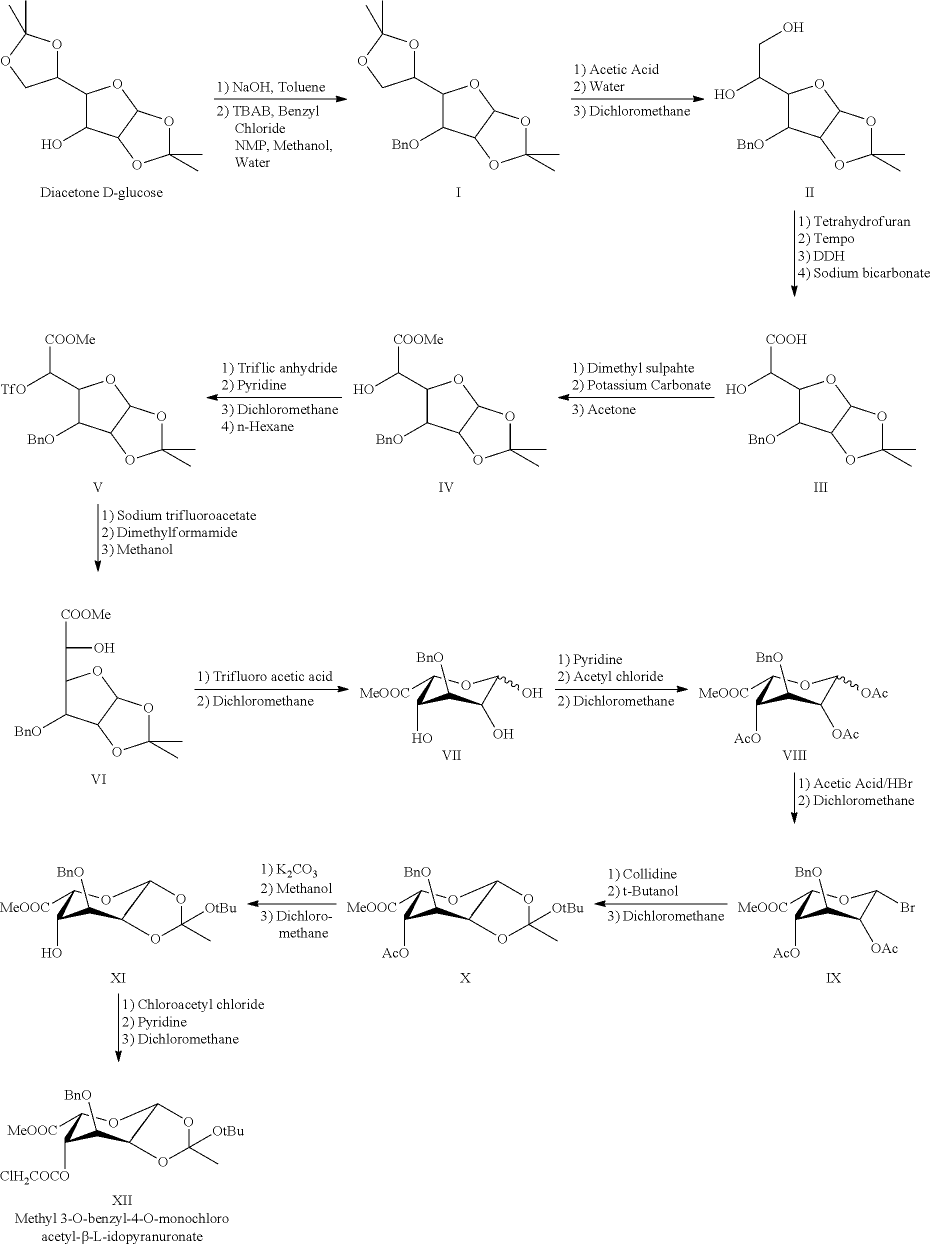

### Route of Synthesis for α-Methyl-6-o-acetyl-3-o-benzyl-2-(benzyloxy carbonyl)amino-2-deoxy-α-D-glucopyranoside

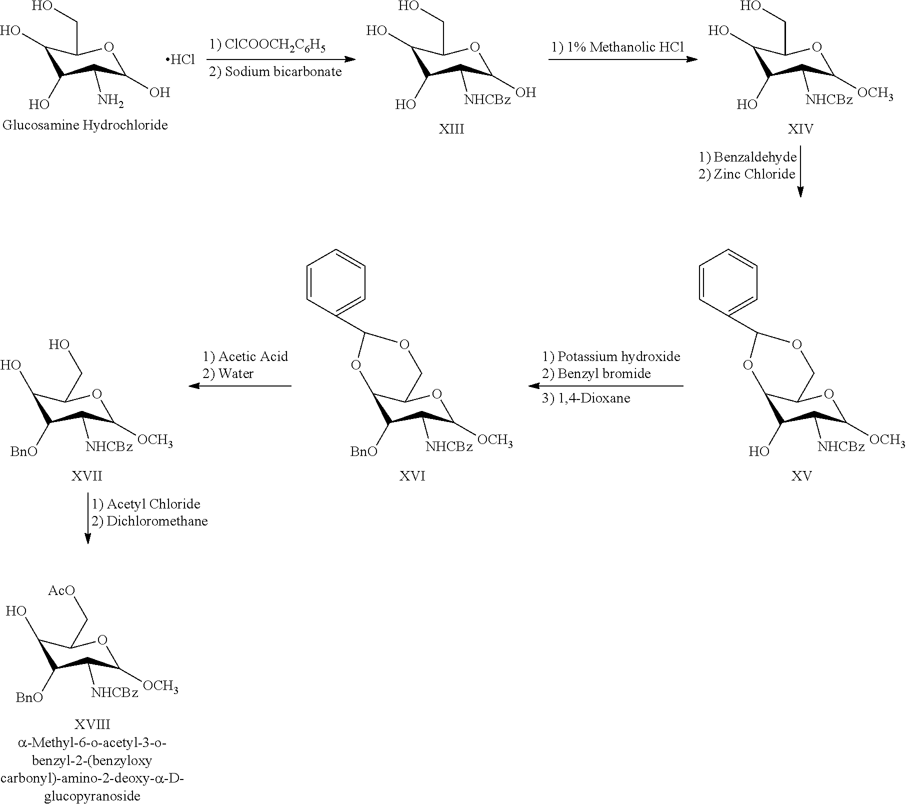

### Methyl 6-O-acetyl-3-O-benzyl-2-(benzyloxy carbonyl)amino-2-deoxy-4-O-(methyl-2-O acetyl-3-O-benzyl-α-L-idopyranosyluronate)-glucopyranoside

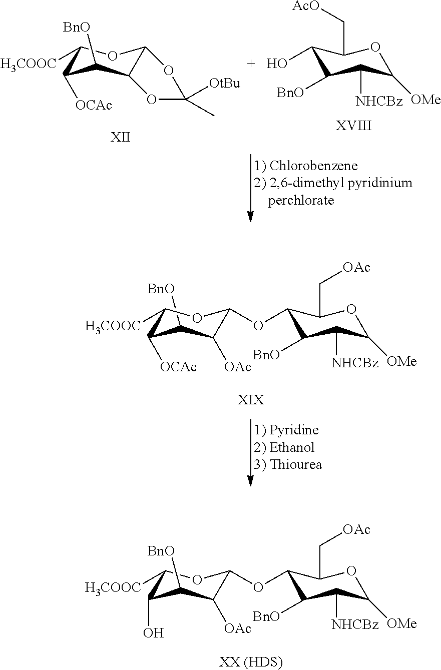

### Route of Synthesis for 1, 6-Anhydro-2-azido-3-O-acetyl-2-deoxy-beta-D-glucopyranose

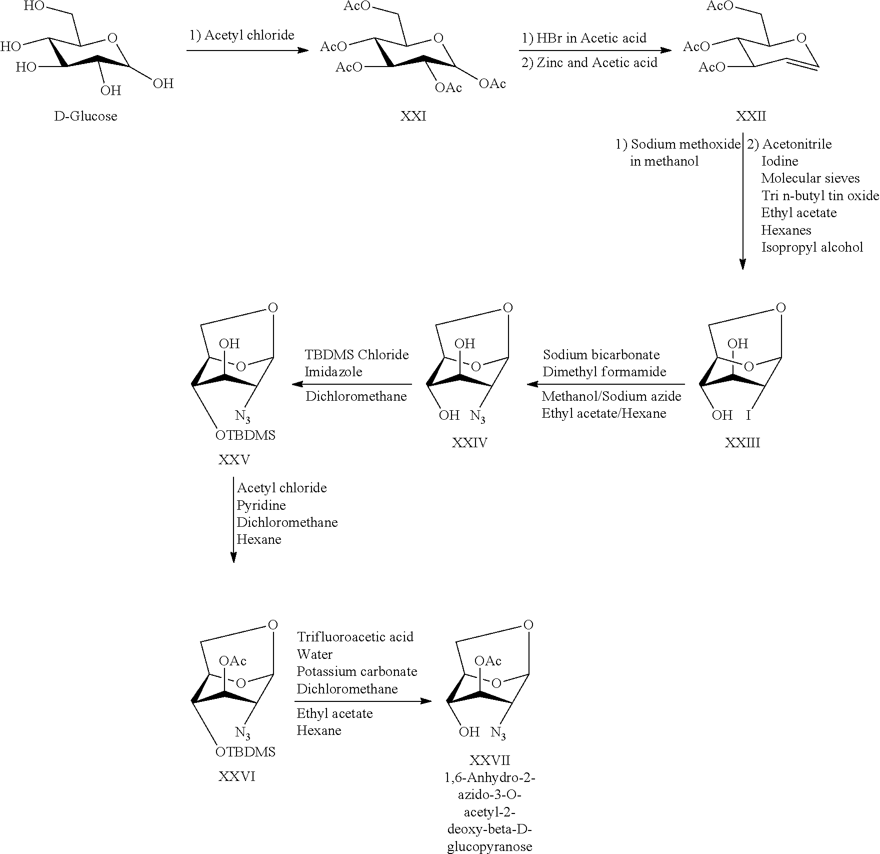

### Route of synthesis for Methyl 2, 3-di-O-benzyl-4-O-chloroacetyl-beta-D-glucopyranuronate

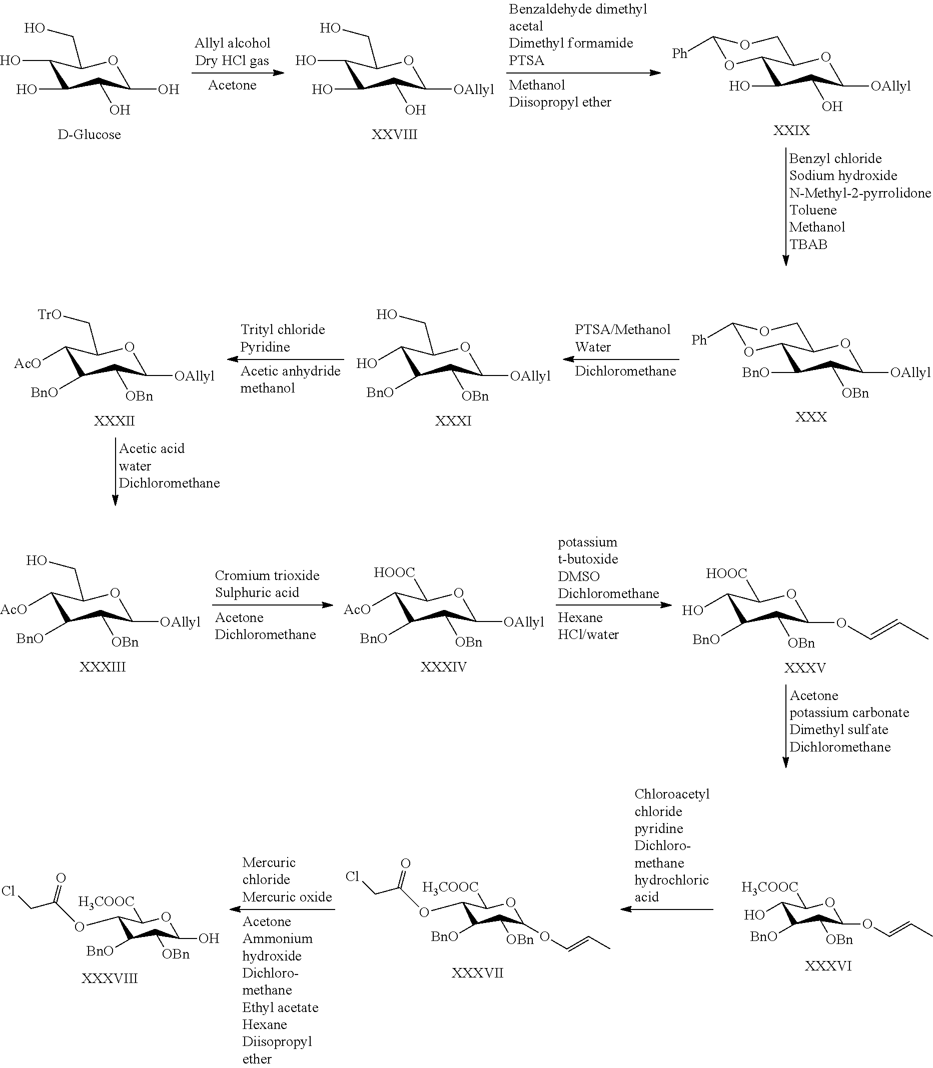

### Route of Synthesis for

### 3-O-Acetyl-1,6-anhydro-2-azido-4-O-2,3-di-O-benzyl-4-O-chloroacetyl-beta-D-glucopyranosyl methyluronate-beta-D-glucopyranose

### or

### 3-O-Acetyl-1,6-anhydro-2-azido-2-deoxy-4-O-(methyl 2,3-di-O-benzyl-4-O-chloroacetyl-beta-D-glucopyranosyluronate)-beta-D-glucopyranose

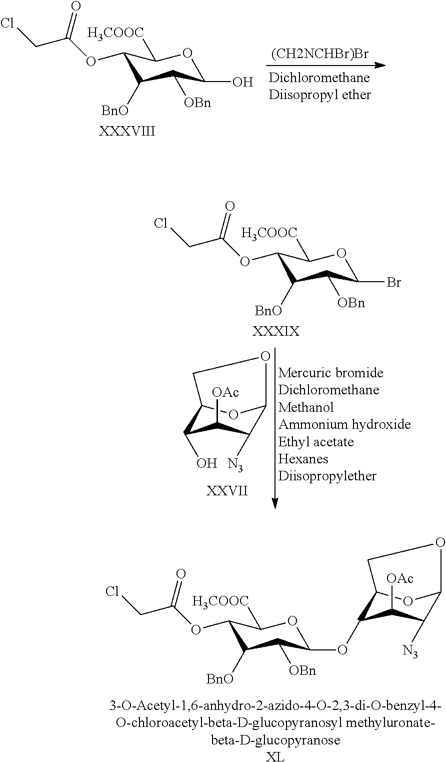

### Route of Synthesis for 1,6-Anhydro-2-azido-3,4-di-O-benzyl-2-deoxy-beta-D-glucopyranose

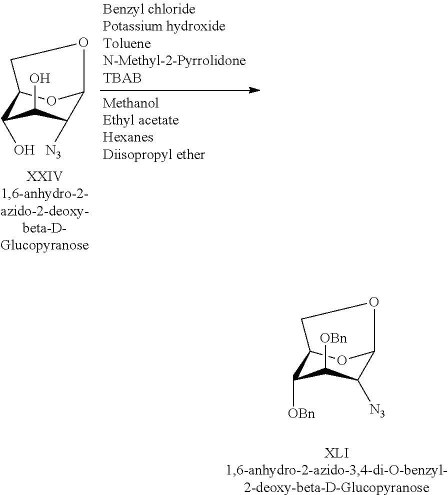 [SMILES: [N-]=[N+]=N[C@H]1C2OC[C@@H](O2)[C@@H](O)C1O ; "XXIV1,6-anhydro-2-azido-2-deoxy-beta-D-Glucopyranose" ; [N-]=[N+]=N[C@@H]1C2OC[C@@H](O2)[C@@H](OCc2ccccc2)C1OCc1ccccc1 ; "XLI1,6-anhydro-2-azido-3,4-di-O-benzyl-2-deoxy-beta-D-Glucopyranose"]

### Synthesis of Disaccharide XLIII

Disaccharide XLIII was prepared in 2 synthetic steps from CADS sugar (XL) using the following procedure:

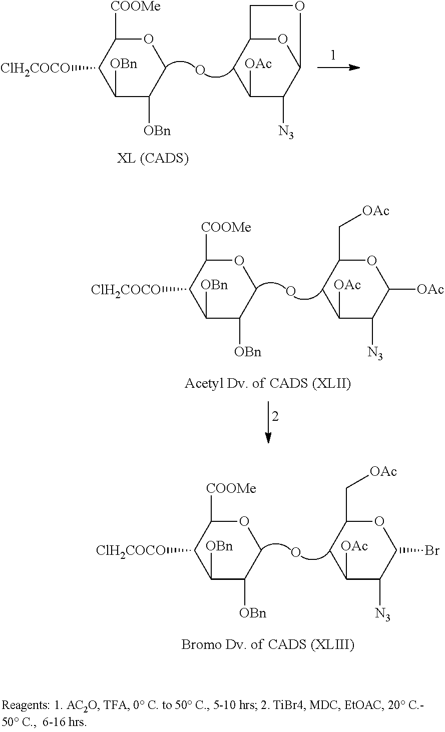

CADS sugar XL was acetylated at the anomeric carbon using AC₂O and TFA to give acetyl derivative XLII. This step was carried out using the reactants CADS, AC₂O and TFA, stirring in an ice water bath for about 5-24 hours, preferably 20 hours, and evaporating to residue under vaccum. Residue was recrystallized in ether. Acetyl CADS (XLII) was brominated at the anomeric carbon using titanium tetra bromide in MDC and ethylacetate and stirring at 20° C.-50° C. for 6-16 hours, preferably 6 hours, to give the bromo derivative, (XLIII) after work-up and recrystalization from solvent/alcohol.

### Synthesis of the Monosaccharide (XLV)

The monosaccharide (XLV) was prepared in 2 synthetic steps from monomer (XLI) using the following procedure:

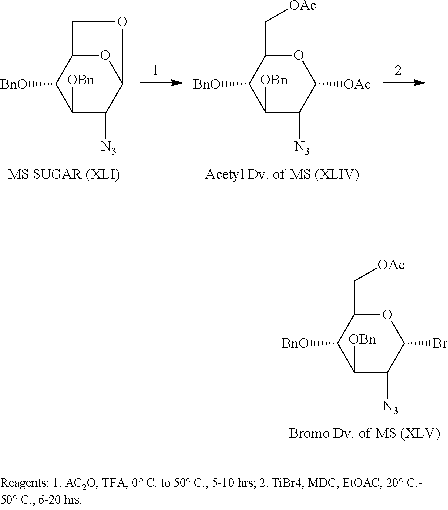 [SMILES: [N-]=[N+]=NC1C2OCC(O2)[C@@H](OCc2ccccc2)C1OCc1ccccc1 ; CC(=O)OCC1O[C@H](OC(C)=O)C(N=[N+]=[N-])C(OCc2ccccc2)[C@@H]1OCc1ccccc1 ; CC(=O)OCC1O[C@H](Br)C(N=[N+]=[N-])C(OCc2ccccc2)[C@@H]1OCc1ccccc1]

Mono sugar (XLI) was acetylated at the anomeric carbon using AC₂O and TFA to give acetyl derivative (XLIV). This step was carried out using the reactants Mono sugar (XLI), AC₂O and TFA, stirring in an ice water bath for about 5-24 hours, preferably 24 hours, and evaporating to residue under vacuum. Residue was recrystallized in ether. Acetyl Mono sugar (XLIV) was brominated at the anomeric carbon using titanium tetra bromide in MDC and ethyl a c et at e and stirring at 20° C.-50° C. for 6-20 hours, preferably 16 hours, to give the bromo derivative, (XLV) after work-up and recrystalization from ether.

Synthesis of the Hydroxy Tetrasaccharide (XLVII)

The hydroxy tetrasaccharide (XLVII) was prepared in 2 synthetic steps from disaccharide (XLIII) and HDS (XX) using the following procedure:

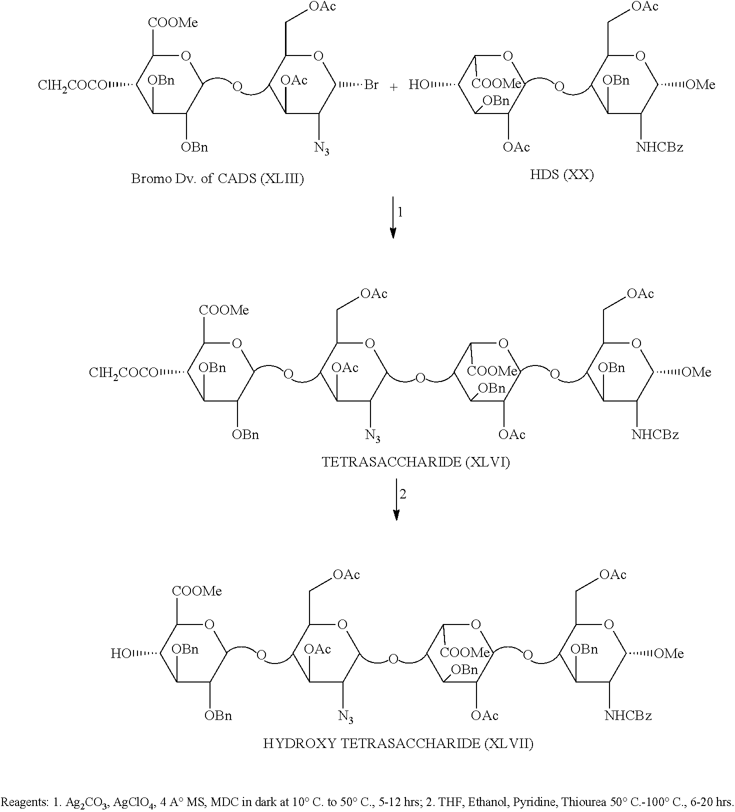

Disaccharide (XLIII), was coupled with disaccharide (XX) in the presence of silver carbonate, silver per chlorate and 4 A° MS in MDC and stirred at ambient temperature for 5-12 hrs, preferably 4-6 hours, in the dark followed by work-up and purification in water/methanol to give the tetrasaccharide (XLVI). The dechloroacetylation of tetrasaccharide (XLVI) was carried out in THF, ethanol and pyridine in the presence of thiourea at reflux for 6 to 20 hrs, preferably 12 hours, to give the hydroxy tetrasaccharide (XLVIII).

Synthesis of the Pentasaccharide (XLVIII)

The pentasaccharide (XLVIII) was prepared in 2 synthetic steps from monosaccharide (XLV) and tetrasaccharide (XLVII) using the following procedure:

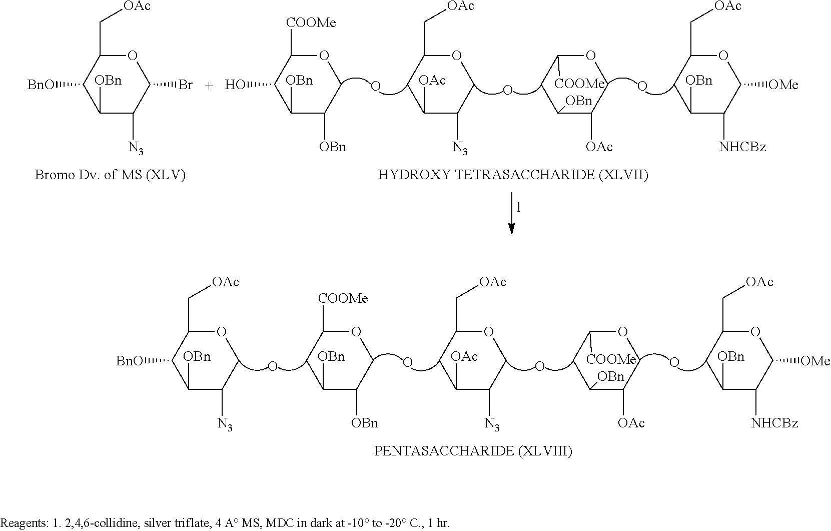

Monosaccharide (XLV), was coupled with tetrasaccharide (XLVII) in the presence of 2,4,6-collidine, silver triflate and 4 A° MS in MDC and stirred at −10° C. to −20° C. for 1 hr in the dark followed by work-up and purification by column chromatography to give the pentasaccharide (XLVIII).

Synthesis of OS Pentasaccharide (L)

The OS pentasaccharide (L) was prepared in 2 synthetic steps from pentasaccharide (XLVIII) using the following procedure:

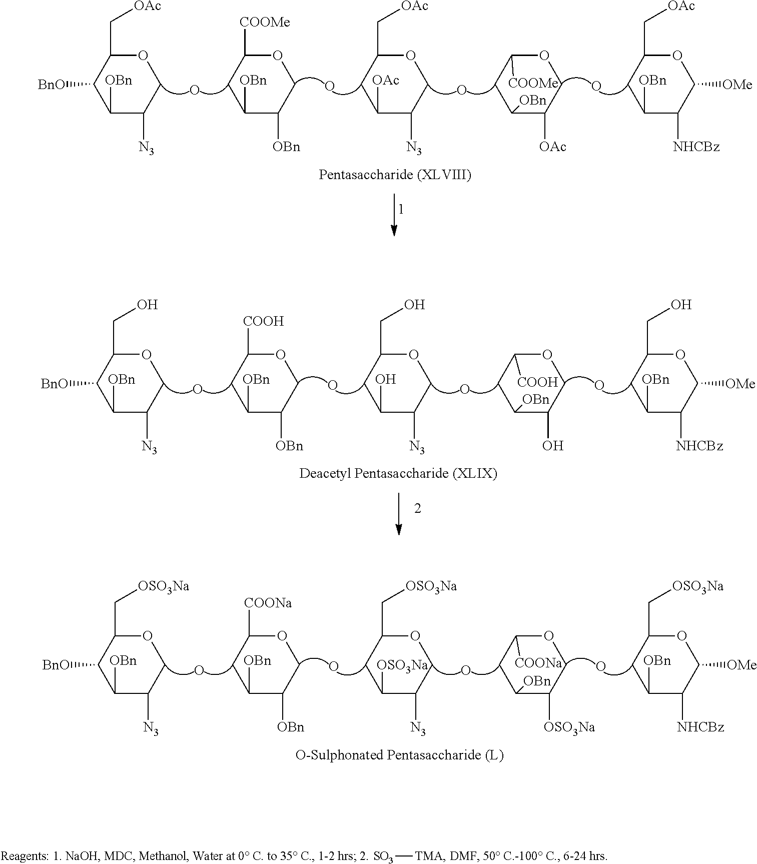

Pentasaccharide (XLVIII) was deacetylated in the presence of NaOH in mixture of solvents of MDC, methanol and water at 0° C. to 35° C., for 1-2 hrs followed by work-up and distillation to obtain deacetylated pentasaccharide (XLIX) which was subjected to O-sulfonation in DMF in the presence of SO₃-trimethylamine (TMA) at 50° C. to 100° C., preferably 50° C.-55° C., for 6-24 hrs, preferably 12 hours, followed by salt removal through Sephadex® resin and column chromatography purification, then pH adjustment by dilute NaOH to give OS pentasaccharide (L).

Synthesis of Fondaparinux Sodium (LIII)

Fondaparinux sodium (LIII) was prepared in 3 synthetic steps from O—S pentasaccharide (L) using the following procedure:

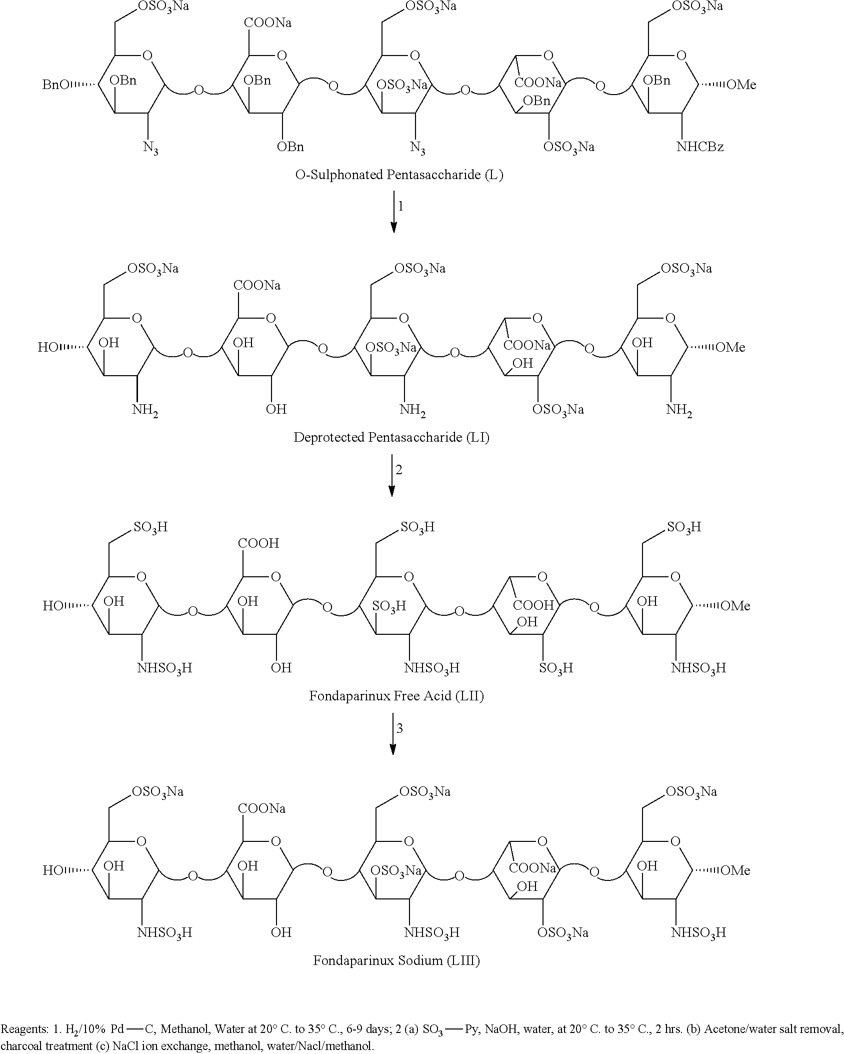

The intermediate L was then hydrogenated to reduce the two azides and N-CBz protection on sugars XLVIII, XX and XLV to amines and the reductive deprotection of the six benzyl ethers to their corresponding hydroxyl groups to form the intermediate deprotected pentasaccharide (LI). This transformation occurs by reacting L with 10% palladium/carbon catalyst with hydrogen gas for 6-9 days, preferably 9 days. The amino groups on deprotected pentasaccharide (LI) were then sulfonated using the pyridine-sulfur trioxide complex in sodium hydroxide, allowing the reaction to proceed for 2 hours to provide fondaparinux free acid (LII) which is purified and is subsequently converted to its salt form. The crude mixture was purified using an ion-exchange chromatographic column (Dowex 1x2-400 resin) followed by desalting using a methanol treatment and purification by water/NaCl/methanol to give the final API, fondaparinux sodium.

### Experimental Procedures

### Preparation of Benzylation of Diacetone-D-Glucose (I)

25 kg of diacetone-D-glucose at RT was charged into a reactor then 250.0 L of toluene, 25 L of NMP followed by 2.5 kg of tetra-n-butylammonium bromide (TBAB) were charged into the reaction mass at RT and the reaction was stirred for 15-20 minutes at RT. Next, 11.5 kg of sodium hydroxide was charged into the reactor and the reaction mass was stirred for 20-25 minutes at RT, then 18.25 kg of benzyl chloride was slowly added into it and the reaction was stirred for 5-7 hrs, preferably 7 hours, at RT then 18.25 L of methanol was charged into the reaction mass and the reaction was stirred for 15-20 minutes at RT. Water work-up and evaporation yielded 21.5 kg of compound (I).

Deprotection of Compound (I)

21.5 kg of compound (I) was charged at RT into the reactor followed by addition of 110 kg of acetic acid and 25 L of water in to the reaction mass at RT and the reaction was stirred for 6-8 hrs at 40° C.-45° C. The reaction mass was cooled down to RT and subjected to two hexane washes and the product was extracted in MDC. The organic layer was again washed with NaHCO₃ solution and brine solution. Evaporation yielded 19.0 kg of compound (II)

Oxidation of compound (II)

375 L of THF and 19 kg of compound (II) were charged in a reactor with 125 L of water at RT. The reaction mass was cooled to 0° C.-−5° C.40 kg of NaHCO₃, 27.5 kg of dichlorodimethylhydantoin (DDH) and 187.5 gm of tetramethylpiperidinol N-oxyl (TEMPO) were added into the reaction mass. The reaction mass was stirred for 6-8 hrs at 0° C.-−5° C. then diluted with sodium thiosulphate solution, washed with hexane and the pH of the aqueous layer was adjusted to 2-3 with HCl solution and the product extracted with MDC. The organic layer was washed with water then brine solution, dried over sodium sulfate, and after evaporation yielded 17.50 kg of compound (III).

Esterification of compound (III)

127.5 L of acetone was charged at RT into a reactor, then 17 kg of compound (III) was charged into the reaction mass at RT and the reaction was stirred for 5-10 minutes at RT. 23.5 kg of potassium carbonate was then added and the reaction was stirred for 10-15 minutes at RT, then 7.31 kg of dimethyl sulphate was slowly added into it and the reaction was stirred for 1-2 hrs at RT. 382.5 L of water and 68 L of MDC was then added and the reaction mass was stirred for 10-15 minutes at RT. Separated layers. After further extraction of aqueous layer with MDC, finally the organic layer was washed with water and dried over sodium sulfate. After evaporation the yield was 12.3 kg of compound (IV).

O-Protection of Compound (IV)

36 L of MDC and 12 kg of compound (IV) were charged in a reactor at RT under nitrogen atmosphere and the reaction mass was cooled to −30° C.-−35° C., then 4.2 kg of pyridine were slowly added. The reaction mass was again cooled to −45° C.-−50° C., then 10.56 kg of triflic anhydride was slowly added into it. The reaction mass was stirred for 15-30 minutes at −45° C.-−50° C., then the reaction mass was quenched into hexane and filtered. The clear filtrate was dried over sodium sulfate, and after evaporation yielded 10.4 kg of compound (V).

Deprotection and Isomerisation of Compound (V)

10.4 kg of compound (V) was charged at RT into a reactor then 36 L of DMF and 14.40 kg of sodium TFA was charged into the reaction mass at RT and the reaction was stirred for 2-3 hrs at 75° C.-80° C., then the reaction mass was cooled down to RT. After MDC/water work-up and evaporation yielded 9.8 kg of product. It was stirred with methanol at RT for 12 hrs then distilled off completely to yield 7.2 kg of compound (VI).

Deprotection and ring expansion of compound (VI)

29.05 kg of TFA was charged into a reactor then cooled to 10° C.-15° C. 2.1 L of water and compound (VI) were charged slowly into the reaction mass at 10° C.-15° C. and the reaction was stirred for 1-2 hrs at 10° C.-15° C. The reaction mass was quenched in water and MDC, the pH of the aqueous layer was adjusted to 7.5-8.5 with potassium carbonate solution. Both organic and aqueous layers were separated and the aqueous layer was extracted twice with MDC. All organic layers were dried over sodium sulfate, and after evaporation yielded 4.75 kg of compound (VII).

Acetylation of Compound (VII)

18.45 kg of pyridine and 4.5 kg of compound (VII) were charged into a reactor then cooled to 0° C.-5° C. 8.32 kg of acetyl chloride was charged slowly into the reaction mass at 0° C.-5° C. The reaction mass temperature was raised to RT and the reaction was stirred for 8-10 hrs at RT. The reaction mass was diluted with water/MDC, extracted with MDC and slowly the pH of the reaction mass adjusted to 1-2 with HCl solution. The organic layer was washed with water, dried over sodium sulfate, and after evaporation, the residue was purified in a silica column using the following gradient profiles: 20:80 to 30:70 (EtOAc/hexane). The pure fractions were pooled and evaporated to yield 1.35 kg of compound (VIII).

### Bromination and Orthoesterification of Compound (VIII)

6 L of MDC and 8.4 kg of HBr in acetic acid were charged into a reactor under nitrogen atmosphere, then cooled to −5° C.-5° C. A solution of 1.2 kg of compound (VIII) in MDC was slowly added into the reaction mass at −5° C.-5° C. The reaction was stirred for 2 hrs at −5° C.-5° C., the reaction mass was quenched in cold water, and the pH of the reaction mass was adjusted to 7.0-8.0 with sodium bicarbonate solution. The organic and aqueous layers were separated. The organic layer was washed with brine solution, dried over sodium sulfate, and after evaporation, the reaction mass was cooled to RT. 0.24 kg of 4 A° MS was then charged into reactor under nitrogen atmosphere. A solution of 1.56 L of collidine and 1.8 L of t-butanol in MDC was slowly charged into the reaction mass at RT. The reaction was stirred for 12 hrs at RT then the reaction mass was quenched into water and filtered. Organic and aqueous layers were separated and the pH of the organic layer was adjusted to 4-4.5 with potassium bisulphate. The organic and aqueous layers were separated again and then adjusted to 7.0-8.0 with NaHCO₃ solution. Organic and aqueous layers were separated and the organic layer was washed with brine solution, dried over sodium sulfate, and after evaporation, the residue was purified in a silica column using the following gradient profiles: 20:80 to 30:70 (EtOAc/hexane). The pure fractions were pooled and evaporated to yield 0.62 kg of compound (X).

Deacetylation of Compound (X)

3.0 L of methanol, 0.12 kg of 4 A° MS and 0.6 kg of compound (X) were charged into a reactor under nitrogen atmosphere then cooled to −20° C. to −25° C. The reaction was stirred for 3-4 hrs at −20° C. to −25° C., the reaction mass was diluted with MDC and filtered through Celite® filter, and washed with water. Theorganic layer was washed with brine solution, dried over sodium sulfate, and after evaporation the yield was 0.4 kg of compound (XI).

Chloroacetylation of compound (XII)

3.0 L of MDC and 0.4 kg of compound (XI) were charged into a reactor under nitrogen atmosphere then cooled to 0° C.-5° C. 0.48 L of pyridine was charged into the reactor then cooled to −20° C. to −25° C. A solution of 0.2 kg of CAC in MDC was slowly charged into the reaction mass at −20° C. to −25° C. The reaction was stirred for 20-30 minutes at −20° C. to −25° C. The reaction mass was diluted with MDC and quenched into cold water. The organic and aqueous layers were separated and the organic layer was washed with KHSO₄ solution, NaHCO₃ solution and brine solution, and dried over sodium sulfate. After evaporation, the residue was purified in a silica column using the solvent system: 20:80:1 (EtOAc/hexane/TEA). The pure fractions were pooled and evaporated to yield 0.35 kg of compound (XII).

### N-Protection of Glucosamine Hydrochloride

A solution of 11.7 kg of NaHCO₃ in 130 L water at RT was charge into a reactor. 10 kg of glucosamine hydrochloride was then charged into the reaction mass at RT and the reaction was stirred for 25-30 minutes at RT. 9.5 kg of benzyl chloroformate was slowly charged into the reaction mass at RT and the reaction was stirred for 3 hrs at RT and filtered. Wet product was treated with water and methanol to yield 9.1 kg of compound (XIII).

O-Methylation of compound (XIII)

124 L of 1% methanolic HCl and 9.0 kg of compound (XIII) at RT were charged into a reactor and the reaction was stirred for 14 hrs at 60° C.-65° C. The reaction mass was cooled down to RT, and 1.8 kg of NaHCO₃ was slowly added into the reaction mass to maintain the pH between 6.5-7.5. The reaction mass was cooled down to 0° C.-5° C., the reaction was stirred for 20-25 minutes at 0° C.-5° C. then filtered. After evaporation, the residue was stirred with hexane for 1 hr at RT and solid product was isolated by filtration yielding 6.3 kg of compound (XIV).

### O-Protection of Compound (XIV)

30 kg of benzaldehyde were charged at RT into a reactor, then 6 kg of compound (XIV) were charged into the reaction mass at RT and the reaction was stirred for 15-20 minutes at RT. 2.7 kg of zinc chloride was charged into the reaction mass at RT and the reaction was stirred for 24 hrs at RT. 30 L of methanol was charged into the reactor and the reaction mass was stirred for 15-20 minutes. The reaction mass was cooled down to 0° C.-5° C., the reaction was stirred for 45-60 minutes at 0° C.-5° C., and solid product was isolated by filtration to yield 4.2 kg of compound (XV).

### O-Benzylation of Compound (XV)

40 L of 1,4 dioxane and 4.0 kg of compound (XV) were charged at RT into a reactor, then the reaction was stirred for 15-20 minutes at RT. 1.6 kg of KOH and 3.2 kg of benzyl bromide were slowly added into the reactor at RT, the reaction was stirred for 15-30 minutes at RT, then the reaction was refluxed for 4 hrs. The reaction mass was cool down to RT, water was slowly added into the reaction mass, the reaction was stirred for 2 hrs at RT and solid product was isolated by filtration yielding 3.3 kg of compound (XVI).

O-Deprotection of compound (XVI)

9 kg of acetic acid, 3 kg of compound (XVI) and 6 L of water were charged into a reactor at RT and the reaction was stirred for 15-20 minutes at RT. The reaction was stirred for 3-4 hrs at 90° C.-100° C., the reaction mass was cool down to RT, 15 L of water was slowly added into the reaction mass at RT and the reaction was stirred for 10-15 minutes at RT. Solid product was isolated by filtration yielding 1.65 kg of compound (XVII).

### Acetylation of Compound (XVII)

4.5 kg of MDC, 1.5 kg of compound (XVII) and 1.05 kg of pyridine were charged into a reactor and then cooled to −50° C. to −55° C. 0.36 kg of acetyl chloride was charged slowly under nitrogen atmosphere in to the reaction mass at −50° C. to −55° C. The reaction was stirred for 30 minutes at −50° C. to −55° C., the temperature of the reaction mass was raised to 0° C.-5° C., the reaction mass was worked up with water/MDC, extracted with MDC and the pH of the reaction mass slowly adjusted to 2-3 with HCl solution. The organic layer was washed with NaHCO₃ and water at 0° C.-5° C. and dried over sodium sulfate. After evaporation, the residue was purified in EtOAc/hexane to yield 0.75 kg of compound (XVIII).

### Condensation of Monosaccharide (XII) and Monosaccharide (XVIII)

4.5 L of chlorobenzene, 0.3 kg of monosaccharide (XVIII) and 0.039 kg of pyridinium perchlorate were charged into a reactor and the reaction mass was heated to 125° C.-130° C. Water was removed by azeotrophic distillation; the reaction was stirred for 1 hr at 125° C.-135° C. A solution of 0.30 kg of monosaccharide (XII) in chlorobenzene was charged slowly in to it, then the reaction was stirred for 2-3 hrs at 125° C.-135° C. The reaction mass was cooled down to 80° C.-85° C. and the solvent distilled off completely to yield 0.35 kg of Disaccharide (XIX).

### Preparation of HDS-(XX)

1.2 L of methanol, 1.8 L of pyridine, 0.35 kg of disaccharide (XIX) and 0.06 kg of thiourea were charged into a reactor and heated to 90° C.-10 0° C. and stirred for 1 hr at 90° C.-100° C. The reaction mass was cooled down to RT and worked up with water/MDC, extracted with MDC, and the organic layer was washed with KHSO₄, NaHCO₃ and brine solution, and dried over sodium sulfate. After evaporation, the residue was purified in a silica column using the solvent system: 30:70 (EtOAc/hexane). The pure fractions were pooled and evaporated to residue which was purified in EtOAc/DIPE, yielding 0.110 kg of HDS(XX).

Preparation of Compound (XXII) from D (+) Glucose

427.5 kg of acetyl chloride and 150 kg of D (+) glucose were charged into a reactor and cooled to 0° C.-5° C. A solution of 13.5 ml of acetic acid and 1.5 ml of H₂SO₄ was charged slowly into the reaction mass at −0° C.-5° C. The reaction was stirred for 30 minutes at 0° C.-5° C., and the temperature slowly raised to RT, then to 70° C.-75° C. The reaction was stirred for 2 hrs at 70° C.-75° C., then the reaction mass was cooled down to RT. 450 kg of HBr in acetic acid was charged slowly into the reaction mass at RT. The reaction was stirred for 2 hrs at RT. Separately, 675 L of water and 450 kg of sodium acetate trihydrate were charged into a reactor. To this reactor a solution of 22.5 kg of copper sulphate in water was added slowly, then cooled to 0° C. to −5° C. 195 kg of zinc dust and 435 kg of AcOH were added into the reaction mass at 0° C. to −5° C. To this reaction mass, the above brominated R/M was slowly charged at 0° C. to −5° C., then cooled to 0° C. to −5° C. The reaction was stirred for 2 hrs at 0° C. to −5° C. then filtered through Celite® filter and worked up with water/MDC, extracted with MDC and the organic layer was washed with NaHCO₃ and water, and dried over sodium sulfate. After evaporation, the residue was purified in IPA to yield 68 kg of compound (XXII).

### Preparation of Compound (XXIII)

1406 L of methanol and 125 kg of compound (XXII) were charged into a reactor and cooled to 5° C.-10° C. The pH of the reaction mass was slowly adjusted to between 9-9.5 with sodium methoxide solution at 5° C.-10° C. The reaction was stirred for 3-4 hrs at RT then cooled to 5° C.-10° C. The pH of the reaction mass was adjusted to between 6.5-7.5 with AcOH solution in methanol at 5° C.-10° C. and the solvent was distilled off completely, then cooled to RT. 200 L of acetonitrile, 181.25 kg of 4 A° MS and 200 kg of Bis (tis-n-butyl tin) oxide was charged into the reactor and the reaction was heated to reflux refluxed for 5 hrs. The reaction mass was cooled down to 0° C.-5° C. 173.5 kg of iodine was charged slowly into the reaction mass at 0° C.-5° C. The reaction was stirred for 3-4 hrs at RT then filtered through Celite® filter, the solvent was distilled off completely and worked up with hexane/sodium thiosulphate solution and then extracted with EtOAC and dried over sodium sulfate. After evaporation, the residue was purified in IPA to yield 26 kg of compound (XXIII).

### Preparation of Compound (XXIV)

250 L of DMF, a solution of 0.95 kg of NaHCO₃ in water, 18 kg of sodium azide and 25 kg of compound (XXIII) were charged into a reactor. The reaction was stirred for 10-12 hrs at RT then heated to 118° C.-12 2° C. and stirred for 2-3 hrs at 118° C.-12 2° C. The reaction mass was cooled down to 40° C.-50° C. and 150 L of methanol was charged into it. The reaction was stirred for 20-30 minutes then filtered. After evaporation, the residue was dissolved in EtOAC and filtered. Clear filtrate was distilled off completely and the EtOAC treatment repeated one more time. The residue was purified in a silica column using the gradient profiles: 20:80 to 50:50 (EtOAc/hexane). The pure fractions were pooled and evaporated to yield 10.60 kg of compound (XXIV).

### Preparation of Mono Sugar (XLI)

25 L of toluene, 2.5 kg of compound (XXIV), 2.5 L of N-methylpyrrolidone (NMP) and 0.25 kg of TBAB were charged into a reactor. The reaction was stirred for 10-15 minutes at RT then 3.75 kg of KOH was charged into it and the reaction cooled to 0° C.-5° C. 5 kg of benzyl chloride was added slowly at 0° C.-5° C. The reaction w as stirred for 4-6 hrs at RT and 5 lit of methanol was charged into the reactor. The reaction was stirred for 20-30 minutes then 12.5 lit of water was added. The organic layer was washed with water, dried over sodium sulfate, and after evaporation, the residue was dissolved in EtOAC and filtered. Clear filtrate was distilled off completely and the EtOAC treatment was repeated one more time. The residue was purified in a silica column using the gradient profiles: 0:10 to 10:90 (EtOAc/hexane). The pure fractions were pooled and evaporated to residue which was purified in DIPE to yield 1.8 kg of Mono sugar (XLI).

### Preparation of Compound (XXIX)

Charge 2.0 kg of allyl alcohol in a round bottom flask (RBF) at ambient temperature and cool to 0-5° C. Pass dry HCl gas (0.06 kg) into the reaction mass at 0-5° C. Charge 1.0 kg of D(+) glucose into the RBF at 0-5° C. Slowly raise the reaction mass temperature to 70-75° C. Maintain the reaction mass temperature at 70-75° C. for 5 hrs. Cool the reaction mass to ambient temperature. Adjust the pH to 8.0-9.0 by adding ammonia solution at ambient temperature. Distill off allyl alcohol from the reaction mass. Cool the reaction mass. Charge 0.5 L of acetone into the reaction mass. Distill off solvent and charge 2.0 L of acetone into the reaction mass. Raise the reaction mass temperature to 50-55° C. Stir for 30-45 minutes. Settle the reaction mass for 45-60 minutes. Separate the layers. Charge the bottom layer in the RBF and extract with acetone three more times. Charge all organic layers in the RBF. Distill off solvent completely under vacuum at or below 50° C. Cool the reaction mass to ambient temperature. Charge 0.20 L of dimethyl formamide into the reaction mass, stir the reaction mass for 30-45 minutes. Distill off solvents. Charge 3.0 L of dimethyl formamide into the reaction mass. Stir the reaction mass for 15-20 minutes. Charge 0.674 kg benzaldehyde dimethyl acetal and p-toluene sulfonic acid into the reaction mass. Raise the reaction mass temperature to 100-105° C. Apply low vacuum and maintain the reaction mass for 2 hrs at 100-105° C. under mild vacuum. Distill off solvents completely and cool the reaction mass to 30-40° C. Charge 0.50 L of methanol. Distill off solvent completely. Charge 0.70 L of methanol into the reaction mass and raise the reaction mass temperature to reflux for 25-30 minutes. Cool the reaction mass to 0-5° C. Filter the reaction mass and wash the cake with 0.10 L of methanol. Dry the product for 5 hrs. Yields 0.3 kg of compound (XXIX).

### Preparation of Compound (XXX)

Charge 10.0 L of toluene into a RBF at ambient temperature into a reactor vessel. Charge 1.0 kg of compound (XXIX) into RBF at ambient temperature. Charge 1.0 L of N-methyl-2-pyrrolidone and 0.10 kg of tetra butyl ammonium bromide (TBAB) into the reaction mass at ambient temperature. Stir the reaction mass for 15-20 minutes. Slowly charge 0.65 kg of sodium hydroxide into the reaction mass at ambient temperature. Stir the reaction mass for 15-20 minutes. Slowly add 1.25 kg of benzyl chloride into the reaction mass at ambient temperature over a period of 1-2 hrs. Maintain the reaction mass for 10-12 hrs at ambient temperature. Add 0.75 L methanol into the reaction mass. Add 4.0 L of water in reaction mass; raise the temperature of reaction mass to 40-45° C. Stir the reaction mass for 15-20 minutes at 40-45° C. Separate the layers. Extract the aqueous layer with 10.0 L toluene. Organic layer wash with water to get neutral pH. Charge the organic layer in RBF and distill off solvent completely under vacuum at or below 50° C. Add 6.0 L methanol into the reaction mass then cool the reaction mass to ambient temperature, stir for 1-2 hrs. Filter the product and wash with methanol. Unload the product and dry it. Dry weight=1.1 kg of compound (XXX).

### Preparation of Compound (XXXI)

Charge 1.0 kg of compound (XXX) and 10.0 L of methanol in a RBF at ambient temperature. Add a solution of p-toluene sulfonic acid in water into reaction mass. Raise the temperature of the reaction mass to 70-75° C. and maintain it for 1-2 hrs. Distill off the solvent and cool the residue. Add water and dichloromethane to the residue and separate the layers. Wash the organic layer with water. Distill off the solvent completely to get residue. Weight of residue=0.70 kg of compound (XXXI).

### Preparation of Compound (XXXII)

Charge 1.0 kg of compound (XXXI) and 4.0 L of pyridine in a RBF at ambient temperature. Add 0.95 kg of trityl chloride into the reaction mass. Slowly raise the temperature of the reaction mass to 80-85° C. and maintain the temperature for 2-3 hrs at 80-85° C. Cool the reaction mass to 50-55° C. and add 0.50 kg of acetyl chloride into the reaction mass. Maintain the temperature for 1-2 hrs at 55-60° C. Distill off pyridine completely to get residue. Add 6.0 L of methanol to the residue and cool to 5-10° C. Stir the reaction mass for 1-2 hrs at 5-10° C. and filter the product. Dry the product. Dry weight=1.20 kg of compound (XXXII).

### Preparation of Compound (XXXIII)

Charge 1.0 kg of compound (XXXII), 0.50 L of dichloromethane, 2.0 L of water and 8.40 kg of acetic acid in a RBF at ambient temperature. Raise the temperature of the reaction mass to 40-45° C. Maintain the reaction mass for 6-7 hrs. at 40-45° C. Quench the reaction mass with water. Filter the solid and charge the reaction mass into RBF. Extract the reaction mass with dichloromethane, wash the organic layer with water. Distill off solvent completely to get residue. Weight of residue=0.50 kg of compound (XXXIII).

### Preparation of Compound (XXXIV)

Charge 1.0 kg of compound (XXIII) and 5.0 L of acetone in a RBF. Add Jones reagent in reaction mass at ambient temperature (exothermic reaction). Maintain the reaction mass for 30-45 minutes at 40-45° C. Cool the reaction mass to 15-20° C. and quench the reaction mass with water. Extract the reaction mass with dichloromethane. Wash the organic layer with water and dry using sodium sulfate. Distill off solvent completely to get residue. Residue weight=0.90 kg of compound (XXXIV).

### Preparation of Compound (XXXV)

Charge 3.0 L of dimethyl sulfoxide and 0.98 kg of potassium t-butoxide into a RBF. Raise the reaction mass temperature to 95-100° C. Prepare a solution of compound (XXXIV) in dimethyl sulfoxide (1.0 kg of compound (XXXIV) in 2.0 L DMSO). Add this solution to the above reaction mass at 95-115° C. Raise the reaction mass temperature to 118-122° C. and maintain the temperature for 1-2 hrs. Cool the reaction mass to ambient temperature. Quench the reaction mass in water. Filter the reaction mass through Celite® filter bed. Wash the filtrate with hexane. Adjust the pH of the aqueous layer to 2.0-2.5 with conc.HCl and extract with dichloromethane. Was h the organic layer with water and dry using sodium sulfate. Distill off the solvent completely to get residue. Weight of residue=0.70 kg of compound (XXXV).

### Preparation of Compound (XXXVI)

Charge 1.0 kg of compound (XXXV), 5.0 L of acetone, 0.74 kg of potassium carbonate and 0.34 kg of dimethyl sulphate into a RBF. Stir the reaction mass for 2 hrs at 30-40° C. Filter the reaction mass through Celite® filter. Charge the filtrate and distill off solvent completely to get residue. Charge water and dichloromethane into residue. Stir for 15-30 minutes, separate organic layer. Wash the organic layer with water and dry using sodium sulfate. Distill off solvent completely to get residue of compound (XXXVI).

### Preparation of Compound (XXXVII)

Charge 1.0 kg of compound (XXXVI) and 1.0 L of dichloromethane in a RBF. Cool the reaction mass to 0-10° C. Add 0.42 kg of chloro acetyl chloride at 0-10° C. Add 1.80 L of pyridine in reaction mass at 0-10° C. Maintain the reaction mass for 1-2 hrs at 0-10° C. Quench the reaction mass with water and extract the product with dichloromethane. Charge organic layer and water in RBF. Adjust the pH of the reaction mass with concentrated HCl to 2.0-3.0. Separate the organic layer and charge in RBF. Add water in organic layer and adjust the pH of reaction mass with sodium bicarbonate to 7.0-8.0. Separate layers and wash organic layer with water. Dry organic layer using sodium sulfate and filter it. Charge filtrate in RBF and distill off solvent completely to get residue. Residue weight=0.70 kg of compound (XXXVII).

### Preparation of Compound (XXXVIII)

Charge 1.0 kg of compound (XXXVII), 10.0 L of acetone, 5.0 L of water and 1.15 kg of mercuric oxide into a RBF. Stir the reaction mass for 15-30 minutes. Prepare mercuric chloride solution in acetone (1.45 kg mercuric chloride in 9.0 L of acetone). Slowly add this solution into above reaction mass at ambient temperature. Maintain the reaction mass for 30-60 minutes at ambient temperature. Filter the reaction mass through Celite® filter bed and adjust the reaction mass to pH 8.0-9.0. Filter the reaction mass and distill off acetone. Extract aqueous layer with ethyl acetate. Wash organic layer with sodium chloride and dry the organic layer using sodium sulfate. Distill off solvent completely to get residue. Purify the crude product using silica column chromatography with ethyl acetate:hexane.(10:90 to 20:80) A product containing fractions is pulled out and solvent is distilled off completely to get residue. Product crystallized in isopropyl ether. Weight of product=0.20 kg of compound (XXXVIII).

### Preparation of Compound (XXV)

Charge 1.0 kg of compound (XXIV) compound and 10.0 L of dichloromethane in a RBF at ambient temperature. Cool the reaction mass to 15-20° C. Add 0.80 kg of imidazole and 0.97 kg of tert-butyldimethylsilyl ether (TBDMS) chloride into the reaction mass at 15-20° C. Raise the reaction mass temperature to ambient temperature. Maintain the reaction mass for 10-12 hrs at ambient temperature. Quench the reaction mass with water. Wash organic layer subsequently with dilute hydrochloride solution and dilute sodium bicarbonate solution. Distill off solvent completely, then cool the reaction mass to ambient temperature. Weight of product=1.1 kg of compound (XXV).

### Preparation of Compound (XXVI)

Charge 1.0 kg of compound (XXV) and 2.28 kg of pyridine in a RBF at ambient temperature. Add 1.0 kg of acetyl chloride to reaction mass at ambient temperature. Maintain the reaction mass for 5-6 hrs at ambient temperature. Quench the reaction mass with ice cold water. Wash the organic layer subsequently with dilute hydrochloride solution and dilute sodium bicarbonate solution. Distill off solvent completely, then cool the reaction mass to ambient temperature. Charge hexane in residue, cool the reaction mass temperature to 10-15° C. and maintain reaction mass temperature for 20-30 minutes. Filter the product and wash with hexane. Dry the product for 5-6 hrs. Weight of product=0.70 kg of compound (XXVI).

### Preparation of Compound (XXVII)

Charge 4.91 kg of trifluoroacetic acid in a RBF at ambient temperature. Cool the reaction mass to 10-15° C. Slowly add 0.80 L of water into the reaction mass below 20° C. Charge 1.0 kg of compound (XXVI) into reaction mass below 20° C. Maintain the reaction mass for 5-6 hrs at ambient temperature. Charge dichloromethane into reaction mass and adjust the reaction mass pH to 8.0-9.0 with potassium carbonate solution. Extract aqueous layer with dichloromethane. Dry the organic layer using sodium sulfate. Distill off solvent completely to get crude product. Purify product by column chromatography. Run the column with ethyl acetate: hexane (10:90 to 20:80). Charge all product-containing fractions into RBF and distill off solvent completely to get product. Weight of product=0.50 kg of compound (XXVII).

Preparation of Disaccharide (XL)

Charge 1.50 kg of triphenyl phosphine and 5.0 L of dimethyl formamide in a RBF at ambient temperature. Cool the reaction mass to 0-10° C. Slowly add 1.0 kg of bromine into the reaction mass at 0-10° C. Slowly raise the reaction mass temperature to 58-60° C. Maintain the reaction mass for 30-45 minutes at 58-60° C. Cool the reaction mass to ambient temperature and add diisopropyl ether. Filter the product and wash with diisopropyl ether. Charge wet cake of above product in RBF and add 4.0 L of dichloromethane into the RBF. Prepare a solution of compound (XXXVIII) in dichloromethane and slowly add this solution into the above reaction mass at ambient temperature. Maintain the reaction mass for 1.0 hour at ambient temperature. Filter the reaction mass and charge filtrate into RBF. Adjust the reaction mass to a pH of 8.0-9.0 by using sodium bicarbonate solution. Wash the organic layer with water and dry using sodium sulfate. Distill off the solvent completely under vacuum to get residue. Triturate residue with diisopropyl ether to remove unwanted salt. Distill off filtrate completely to get crude compound (XXXIX). Charge 1.50 L of dichloromethane and 0.70 kg of compound (XXVII) into a RBF at ambient temperature. Add 0.15 kg of molecular sieves into the reaction mass at ambient temperature. Stir the reaction mass 15-20 minutes. Slowly add 0.70 kg of mercuric bromide into the reaction mass at ambient temperature. Maintain the reaction mass 6-8 hrs under nitrogen. Prepare compound (XXXIX) solution in dichloromethane. Slowly add the above-prepared compound (XXXIX) solution into the reaction mass under nitrogen over a period of 1-2 hours. Maintain the reaction mass for 10-12 hours at ambient temperature. Filter the reaction mass and quench with ammonia solution. Filter the solid and filtrate wash with water. Dry the organic layer on sodium sulfate and distill off solvent completely to get residue. Triturate the residue with methanol and stir the reaction mass for 1 hour. Filter the solid and wash with methanol. Treat filtrate with water and separate the product layer. Purify the crude product by column chromatography using ethyl acetate: hexane (0:100 to 20:80) Charge all product-containing fractions into a RBF and distill off solvent completely to get residue. Charge ethyl acetate and diisopropyl ether into the residue. Stir the reaction mass for 20-30 minutes. Dry the product for 4-6 hrs. Weight of product=0.20 kg of disaccharide (XL).

### Preparation of Compound (XLII)

100 gm of CADS (XL) was charged at 20° C.-30° C. into a 2.0 lit RBF under nitrogen atmosphere, then 1.0 L of acetic anhydride was charged, followed by 200 ml of TFA, into the reaction mass at RT and the reaction was stirred for 6 hrs at RT. After evaporation, the residue was stirred with DIPE for 1 hr at RT and solid product was isolated by filtration to yield 95.5 gm of XLII.

NMR spectrum confirmed the expected structure.

### Preparation of Compound (XLIII)

95 gm of acetylated CADS (XLII) was charged at 20° C.-30° C. into a 12.0 L RBF under nitrogen atmosphere with 1.9 L MDC followed by 950 ml ethyl acetate at RT. The reaction mass was stirred for 5-10 min. at RT. To this clear solution, 231.45 gm of titanium bromide were added at RT. The temperature of the reaction mass was raised to 40° C.-45° C. and stirred for 6 hrs. Then the reaction mass was diluted with cold water (1.9 L) and 1.5 L of MDC. The reaction mass was stirred for 10-15 min., both layers were separated and the aqueous layer was extracted with 950 ml of MDC. Both organic layers were combined and dried over sodium sulfate. After evaporation, the residue was recrystallized with 950 ml of IPA for 3 hrs at RT. The solid was filtered & washed with IPA, then DIPE, yielding 52 gm of compound XLIII.

NMR spectrum confirmed the expected structure.

### Preparation of Compound (XLIV)

648 gm of Mono sugar (XLI) was charged at 20° C.-30° C. into a 12.0 L RBF under nitrogen atmosphere. Then 6.48 L of acetic anhydride followed by 1.3 L of TFA were charged into the reaction mass at RT and the reaction was stirred for 8-10 hrs at RT. After evaporation, the residue was stirred with DIPE for 1 hr at RT and solid product was isolated by filtration, yielding 550 gm of compound (XLIV).

NMR spectrum confirmed the expected structure.

### Preparation of Compound (XLV)

550 gm of acetylated Mono sugar (XLIV) was charged at 20° C.-30° C. into a 30.0 L reactor under nitrogen atmosphere with 11 L MDC followed by 100 ml ethyl acetate at RT. The reaction mass was stirred for 5-10 min. at RT. To this clear solution, 779 gm of titanium bromide was added at RT. The reaction mass was stirred for 16 hrs, then the reaction mass was diluted with water (11 L) and 5.5 L of MDC. The reaction mass was stirred for 10-15 min. Both layers were separated and the aqueous layer was extracted with 2.75 L of MDC. Both organic layers were combined and dried over sodium sulfate. After evaporation, the residue was recrystallized with 5.5 L of DIPE for 1 hr at RT. The solid was filtered & washed with DIPE, yielding 469.1 gm of compound (XLV).

NMR spectrum confirmed the expected structure.

### Preparation of Tetrasaccharide (XLVI)

346 gm of bromo CADS (XLIII) with 6.92 L of MDC were charged in a 12 L RBF under argon atmosphere at RT with 207 gm of 4 A° MS. Stirred for 5-10 minutes at RT. When the moisture of the reaction mass was less than 0.05%, then 235 gm of HDS (XX) were charged into it at RT. The reaction mass was stirred at RT for 15-30 minutes, then 176 gm of silver carbonate were added followed by 48.4 gm of silver perchlorate anhydrous added into it at RT in the dark. The reaction mass was stirred for 6 hrs then diluted with 2.08 L of MDC and filtered through a Celite® filter bed, then washed with MDC. Clear filtrate was washed with 10% KHSO₄ solution, then process water, dried over sodium sulfate, and after evaporation, the residue was purified with methanol/water to yield 574 gm of tetrasaccharide (XLVI).

### Preparation of Tetrasaccharide (XLVII)

4.22 L of THF, 0.98 L of ethanol, 422 gm of tetrasaccharide (XLVI), 1.3 L of pyridine and 29.5 gm of thiourea were charged in a 12 L RBF at RT and stirred for 10-15 minutes at RT. The temperature of the reaction mass was raised to 70° C.-80° C. and the reaction mass was stirred at 70° C.-80° C. for 12 hrs. The reaction mass was cooled down to 60° C.-65° C., then the solvent was distilled out completely. The residue was dissolved in 2.96 L of MDC and washed with 10% KHSO₄ solution, then brine solution, dried over sodium sulfate, and after evaporation yielded 398 gm of tetrasaccharide (XLVII).

### Preparation of Pentasaccharide (XLVIII)

8.73 L of MDC and 325 gm of 4 A° MS were charged in a 22 L RBF under argon atmosphere at RT. The reaction mass was stirred at RT for 15-30 minutes, then 406 gm of tetrasaccharide (XLVII) and 406 gm of monosaccharide (XLV) were added. The reaction mass was cooled to −10° C. to −20° C., then 223 ml of 2, 4, 6-collidine and 710 gm of silver triflate were added into the reaction. The reaction mass was stirred for 1 hr in the dark at −10° C. to −20° C. then diluted with 4.67 L of MDC and filtered through a Celite® filter bed and washed with MDC. The clear filtrate was washed with 10% KHSO₄ solution then process water, dried over sodium sulfate, and after evaporation, the residue was purified in a silica column using the following gradient profiles: 20:80 to 50:50 (EtOAc/hexane). The pure fractions were pooled and evaporated to give 250 gm of pentasaccharide (XLVIII).

The impure fractions were pooled and evaporated. The residue was purified in a silica column using the following gradient profiles: 20:80 to 50:50 (EtOAc/hexane). The pure fractions were pooled and evaporated to give pentasaccharide (XLVIII).

NMR Spectrum Confirmed the Expected Structure.

### Preparation of Deacetylated Pentasaccharide (XLIX)

1.06 L of MDC and 245 gm pentasaccharide (XLVIII) were charged in a 12 L RBF at RT, then 3.67 L methanol and 1.07 L of water were added and the reaction mass was stirred for 15-30 minutes at RT. Then a solution of NaOH (564 gm in 2.75 L water) was charged into it at RT and the reaction mass was stirred at RT for 2 hrs. The reaction mass was then diluted with 3.22 L of MDC and 3.22 L of water. Then the pH was adjusted with dilute HCl solution, the organic layer separated and the aqueous layer was extracted with 4.9 L of MDC and washed with brine solution, dried over sodium sulfate, and after evaporation, the residue was purified with IPA/EtoAc/hexane, acetone/water and methanol/water yielding 220 gm of deacetylated pentasaccharide (XLIX)

Preparation of O-sulfonated Pentasaccharide (L)

3.7 L of DMF, 370 gm of deacetylated pentasaccharide (XLIX), and 418 gm of SO₃-TMA complex were charged in a 12 L RBF at RT. The temperature of the reaction mass was raised to 50° C.-55° C. and the reaction mass was stirred at 50° C.-55° C. for 12 hrs. The reaction mass was cooled down to 20° C.-30° C. then diluted with 1.85 L of methanol and layered on top of a column packed with Sephadex® LH-20 resin in methanol:MDC (1:1). The column was run with the same solvent system and required product fractions were collected. After evaporation, the residue was purified in a silica column using the following gradient profiles: 0:100 to 100:0 (methanol/MDC). The pure fractions were pooled and evaporated and the residue was again dissolved in 1.22 L of methanol and pH adjusted to 8-10 with dilute NaOH solution. After evaporation the yield was 300 gm of O-sulfonated pentasaccharide (L).

370 ml of DMF, 37 gm of deacetylated pentasaccharide (XLIX), and 41.8 gm of SO₃-TMA complex were charged in a 2 L RBF at RT. The temperature of the reaction mass was raised to 50° C.-55° C. The reaction mass was stirred at 50° C.-55° C. for 12 hrs. The solvent was distilled off completely to get residue then residue dissolved in 200 ml of methanol: MDC (1:1) and layered on top of a column packed with Sephadex® LH-20 resin in methanol: MDC (1:1). The column was run with the same solvent system and the required product fractions collected, and after evaporation, the residue was purified in a silica column using the following gradient profiles: 0:100 to 100:0 (methanol/MDC). The pure fractions were pooled and evaporated and the residue was again dissolved in 120 ml of methanol and pH adjusted to 8-10 with dilute NaOH solution. After evaporation, the yield was 40 gm of O-sulfonated pentasaccharide (L).

Preparation of Deprotected Pentasaccharide (LI)

(a) 760 ml of water, 2.44 L of methanol, 300 gm O-sulfonated pentasaccharide (L) and 225 gm 10% Pd-C were charged in an autoclave at RT, then hydrogen gas pressure was applied up to 20-60 psi and stirred for 24-72 hrs at RT. The catalyst was then removed by filtration and the clear filtrate was distilled off completely. The residue was dissolved in 760 ml of water and 2.44 L of methanol, then 225 gm fresh 10% Pd-C was added in the autoclave at RT and hydrogen gas pressure then applied up to 20-60 psi and stirred for 24-72 hrs at RT. The catalyst was then removed by filtration and the clear filtrate was distilled off completely. The residue was dissolved in 760 ml of water and 2.44 L of methanol, then 225 gm fresh 10% Pd-C was added in the autoclave at RT and hydrogen gas pressure was applies up to 20-60 psi and stirred for 24-72 hrs at RT. The catalyst was then removed by filtration and the clear filtrate was distilled off completely, yielding 145 gm of deprotected pentasaccharide (LI)

(b) A solution of O-sulfonated pentasaccharide (L) in methanol-water (4:0.5 ml) was hydrogenated in the presence of 10% Pt-C(40 mg) for 5 days. UV spectroscopy was used to indicate whether the reaction was complete, the reaction product was then filtered and concentrated. Subsequent methanol purification gave deprotected pentasaccharide (LI).

### Preparation of Fondaparinux sodium (LIII)—N-sulfonation of Deprotected Pentasaccharide (LI) methyl O-2-deoxy-3,6-di-O-sulfo-2-(sulfoamino)-α-D-glucopyranosyl-(1---->4)-O-2-O-sulfo-α-L-idopyranurosyl-(1---->4)-2-deoxy-6-O-sulfo-2-(sulfoamino)-a-D-glucopyranoside,decasodium salt

A solution of deprotected pentasaccharide (LI) (145 gm) in water (2.54 V) was adjusted to a pH of 9.5-10.5 with 1 N NaOH solution. SO3-pyridine complex (156 gm) was added into 3 lots every 15 min, the pH being maintained at 9.5-10.5 by automatic addition of 1 N NaOH. The mixture was stirred for 2 hrs at RT, during this aqueous NaOH (1N solution) was added to maintain pH at 9.5-10.5. After neutralization to pH 7-7.5 by addition of HCl solution, the mixture was evaporated using vacuum. The residue was dissolved in water (1.6 L) at RT, to this solution was added acetone (1.6 L) at RT. The reaction mass was cooled to 5° C.-10° C. and stirred for 1 hr. The solid was filtered and washed with cold acetone: water (1:1). The clear filtrate was distilled off completely under vacuum below 55° C. The residue was dissolved in water (1.6 L) at RT, and to this solution was added acetone(1.6 L) at RT. The mixture was cooled to 5 to 10° C. and stirred for 1 hr. The solid was filtered and washed with cold acetone/water (1:1). The clear filtrate was distilled off completely under vacuum below 55° C. The residue was dissolved in water (0.7 L) and charcoal (40 gm) was added at RT. The mixture was stirred for 30 min at RT then filtered. To the filtrate was added charcoal (40 gm) at RT. The mixture was stirred for 30 min at RT then filtered. To the filtrate was added charcoal (40 gm) at RT. The mixture was stirred for 30 min at RT then filtered. The pH of the clear filtrate was adjusted to 8.0-8.5 with 1N NaOH solution and distilled off completely under vacuum below 55° C. The residue was dissolved in 0.5 M NaCl solution and layered onto a column of Dowex® 1x2-400 resins using a gradient of NaCl solution (0.5 to 10M). The product fractions were combined and distilled off under vacuum below 55° C. up to 1-2 L volume. The solid was filtered off and the clear filtrate was distilled off under vacuum below 55° C. up to slurry stage and subjected to azeotropic distillation with methanol two times. The solid residue was stirred with methanol (2.13 L) at RT for 1 hr and the solid was filtered off and washed with methanol. The wet solid was again stirred with methanol (2.13 L) at RT for 1 hr and the solid was filtered off and washed with methanol. The wet solid was again stirred with methanol (2.13 L) at RT for 1 hr and the solid was filtered off and washed with methanol. The above solid was dissolved in water and the pH adjusted to 4-4.5 with 1N HCl solution and charcoalized three times with 26 gm of charcoal at RT for 15-30 minutes and filtered off To the clear filtrate was added 0.39 kg of NaCl, then methanol was added (35 volume) at RT and the mixture was stirred for 15-30 minutes. The solution was decanted and the sticky mass was stirred with methanol (0.65 L) at RT for 15-30 minutes. The solid was filtered off and dissolved in water, and the pH adjusted to 8-8.5 with 1N NaOH solution. The solution was filtered through 0.45 micron paper & distilled off completely under vacuum below 55° C. The solution was subjected to azeotropic distillation with methanol to give highly pure fondaparinux sodium (97.17 gm) (HPLC purity 99.7%).

### SOR Results

Three batches of product made in accordance with the present processes provided the following stereoisomeric optical rotation results:

Specification: Between +50.0° and +60.0°.

Batch-1=+55.1°

Batch-2=+55.7°

Batch-3=+55.4°.

While the preferred embodiments have been described and illustrated it will be understood that changes in details and obvious undisclosed variations might be made without departing from the spirit and principle of the invention and therefore the scope of the invention is not to be construed as limited to the preferred embodiment.

What is claimed is:

1. A process for the preparation of fondaparinux sodium comprising:
a) providing monomers having the structures (XII), (XVIII), (XXVII), (XXXVIII) and (XLI) 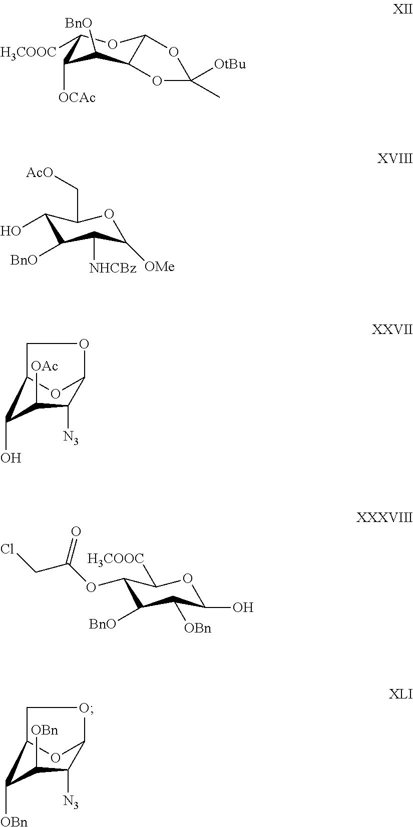
b) linking the monomers having the structures (XII) and (XVIII) to form a disaccharide having the structure (XX) 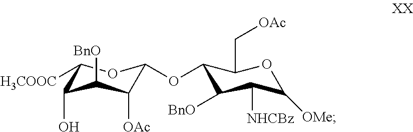
c) linking the monomers having the structures (XXXIX) and (XXVII) to form a disaccharide having the structure (XLIII) 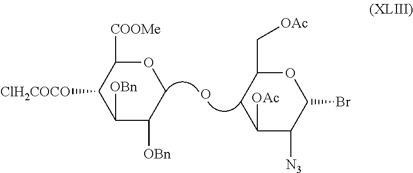
and purifying the disaccharide having the structure (XLIII) by recrystallization;
d) linking the dimers having the structures (XLIII) and (XX) to form a tetrasaccharide having the structure (XLVII) 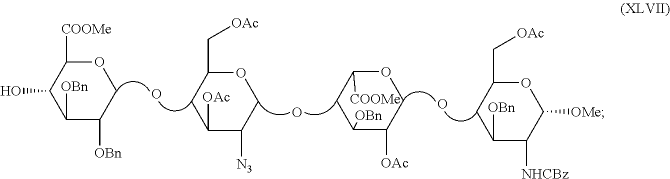
e) providing a monomer having the structure (XLV) 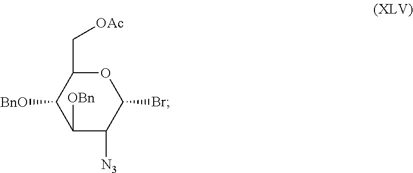 [SMILES: CC(=O)OCC1O[C@H](Br)C(N=[N+]=[N-])C(OCc2ccccc2)[C@@H]1OCc1ccccc1]
and purifying it by recrystallization;
f) linking the monomer having the structure (XLV) to the tetrasaccharide having the structure (XLVII) to form a pentasaccharide having the structure (XLVIII) 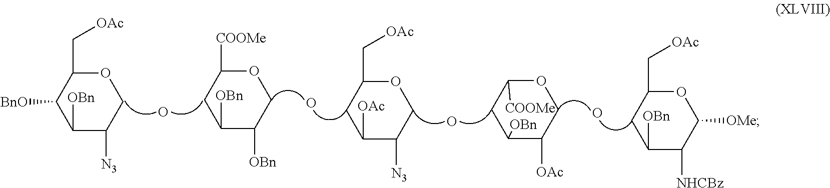
g) converting the pentasaccharide having the structure (XLVIII) by deacetylation to a deacetylated pentasaccharide having the structure (XLIX) 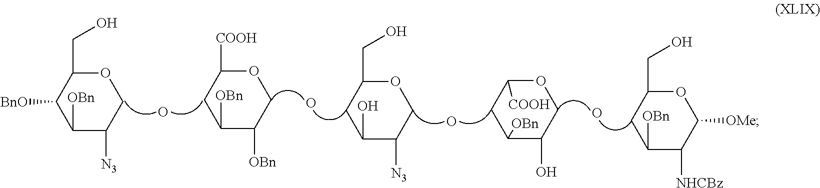
h) subjecting the deacetylated pentasaccharide having the structure (XLIX) to O-sulfonation in DMF in the presence of SO₃-trimethylamine followed by removing the solvent DMF by distillation, salt removal and column purification, then pH adjustment to provide an O-sulfonated pentasaccharide having the structure (L) 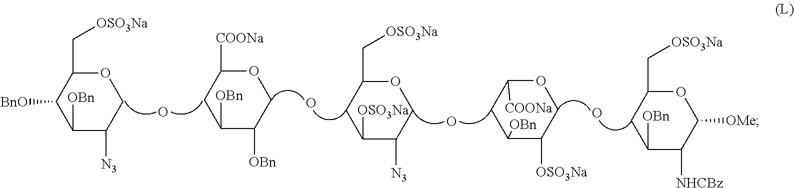
i) converting the O-sulfonated pentasaccharide having the structure (L) to a deprotected pentasaccharide having the structure (LI) 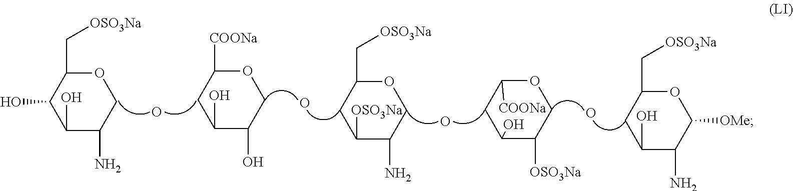
j) converting the deprotected pentasaccharide having the structure (LI) to fondaparinux free acid and purifying the fondaparinux free acid using at least one solvent; and
k) converting the fondaparinux free acid to fondaparinux sodium and purifying the fonadaparinux sodium.

2. The process according to claim 1 wherein the step of providing the monomer having the structure (XII) 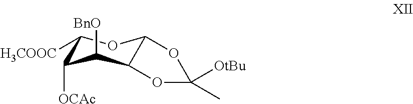
comprises:
a) benzylating diacetone-d-glucose to obtain a benzylated diacetone d-glucose having the structure (I) 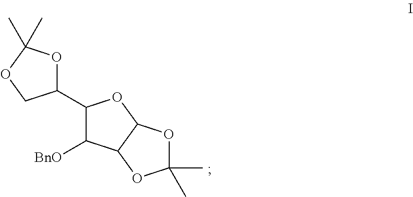 [SMILES: CC1(C)OCC(C2OC3OC(C)(C)OC3C2OCc2ccccc2)O1]
b) deprotecting the benzylated diacetone-d-glucose having the structure (I) to obtain a compound having the structure (II) 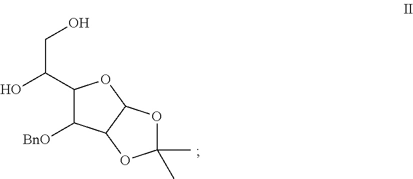 [SMILES: CC1(C)OC2OC(C(O)CO)C(OCc3ccccc3)C2O1]
c) subjecting the compound having the structure (II) to oxidation to obtain a compound (III) 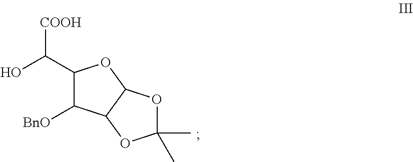 [SMILES: CC1(C)OC2OC(C(O)C(=O)O)C(OCc3ccccc3)C2O1]
d) subjecting the compound having the structure (III) to esterification to obtain a compound (IV) 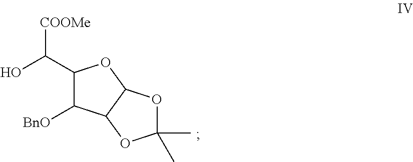 [SMILES: COC(=O)C(O)C1OC2OC(C)(C)OC2C1OCc1ccccc1]
e) treating the compound having the structure (IV) with triflic anhydride and pyridine in an organic solvent to obtain an O-protected compound having the structure (V) 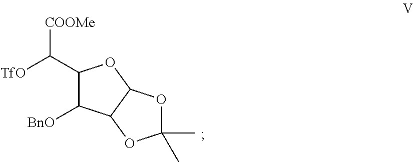 [SMILES: COC(=O)C(OS(=O)(=O)C(F)(F)F)C1OC2OC(C)(C)OC2C1OCc1ccccc1]
f) deprotecting and isomerization of the compound having the structure (V) to obtain a compound having the structure (VI) 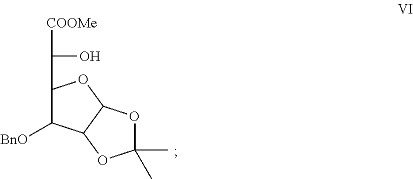 [SMILES: COC(=O)C(O)C1OC2OC(C)(C)OC2C1OCc1ccccc1]
g) deprotecting and performing ring expansion of the compound having the structure (VI) to obtain a compound having the structure (VII) 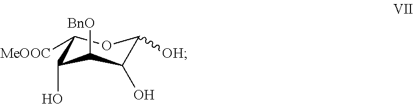
h) acetylating the compound having the structure (VII) to obtain a compound having the structure (VIII) 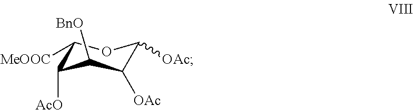
i) brominating the compound having the structure (VIII) to obtain a compound having the structure (IX) 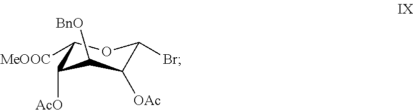
j) performing orthoesterification of the compound having the structure (IX) to obtain a compound having the structure (X) 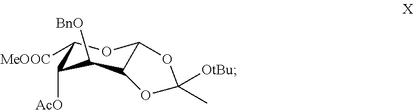
k) deacetylating the compound having the structure (X) to obtain a compound having the structure (XI); 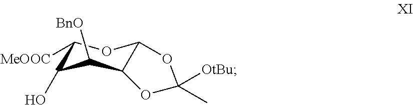
and
l) chloroacetylating the compound having the structure (XI) to obtain the compound having the structure (XII).

3. The process according to claim 1 wherein the step of providing the monomer having the structure (XXVII) 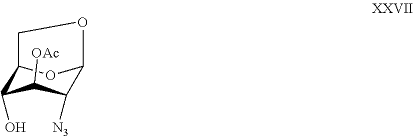 [SMILES: CC(=O)OC1[C@H](N=[N+]=[N-])C2OC[C@@H](O2)[C@H]1O]
comprises
a) acetylating D(+) glucose to produce a compound having the structure (XXI) 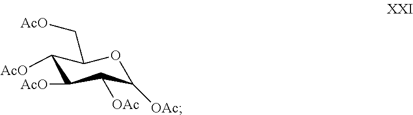
b) subjecting the compound having the structure (XXI) to treatment with HBr and zinc to produce a compound having the structure (XXII) 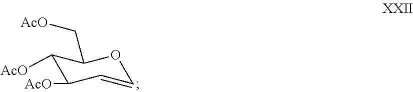 [SMILES: CC(=O)OCC1OC=CC(OC(C)=O)C1OC(C)=O]
c) combining the compound having the structure (XXII) with an organic solvent and subjecting the mixture to treatment with acetonitrile, bis (tis-n-butyl tin) oxide, and iodine to produce a compound having the structure (XXIII) 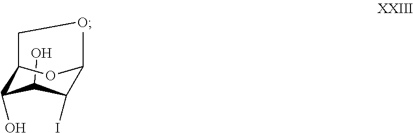 [SMILES: OC1[C@H](I)C2OC[C@@H](O2)[C@H]1O]
d) combining the compound having the structure (XXIII) with dimethylformamide, sodium bicarbonate in water and sodium azide to produce a compound having the structure (XXIV) 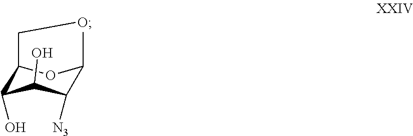 [SMILES: [N-]=[N+]=N[C@@H]1C2OC[C@@H](O2)[C@@H](O)C1O]
e) combining the compound having the structure (XXIV) with dichloromethane, imidazole and tert-butyldimethylsilyl ether (TBDMS) chloride to produce a compound having the structure (XXV) 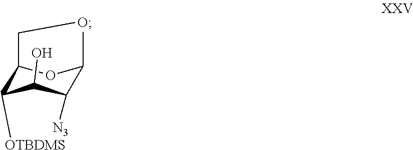 [SMILES: CC(C)(C)[Si](C)(C)O[C@H]1C(O)[C@H](N=[N+]=[N-])C2OC[C@H]1O2]
f) combining the compound having the structure (XXV) with pyridine and acetyl chloride to produce a compound having the structure (XXVI) 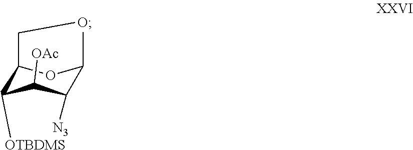 [SMILES: CC(=O)OC1[C@H](N=[N+]=[N-])C2OC[C@@H](O2)[C@H]1O[Si](C)(C)C(C)(C)C]
and
g) combining the compound having the structure (XXVI) with trifluoroacetic acid and subsequently adding dichloromethane, recovering crude product and purifying the product to obtain the compound having the structure (XXVII).

4. The process according to claim 1 wherein the step of providing a monomer having the structure (XLV) 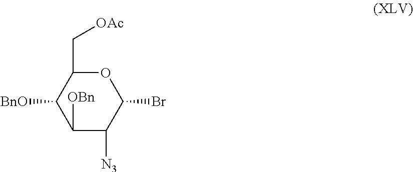 [SMILES: CC(=O)OCC1O[C@H](Br)C(N=[N+]=[N-])C(OCc2ccccc2)[C@@H]1OCc1ccccc1]
comprises combining a compound having the structure (XXIV) with toluene, N-methylpyrrolidone (NMP) and tetra butyl ammonium bromide (TBAB), subsequently adding benzyl chloride, and recovering a monosugar having the structure (XLI) 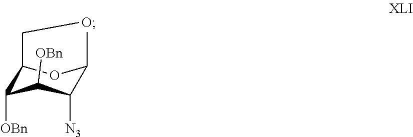 [SMILES: [N-]=[N+]=N[C@@H]1C2OC[C@@H](O2)[C@@H](OCc2ccccc2)C1OCc1ccccc1]
acetylating the monosugar having the structure (XLI) at its anomeric carbon using AC₂O and trifluoroacetic acid (TFA) to produce a compound having the structure (XLIV) 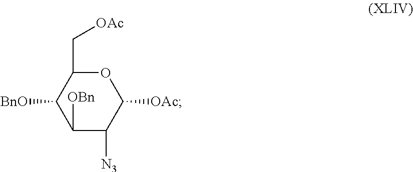 [SMILES: CC(=O)OCC1O[C@H](OC(C)=O)C(N=[N+]=[N-])C(OCc2ccccc2)[C@@H]1OCc1ccccc1]
and
brominating the compound having the structure (XLIV) at the anomeric carbon using titanium tetra bromide in dichloromethane and ethyl acetate to yield the compound having the structure (XLV).

5. A process for making a monomer having the structure (XII) 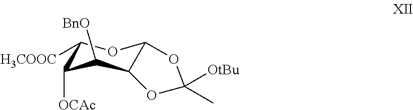
comprising:
a) benzylating diacetone-d-glucose to obtain a benzylated diacetone d-glucose having the structure (I) 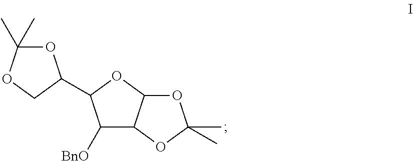
b) deprotecting the benzylated diacetone-d-glucose having the structure (I) to obtain a compound having the structure (II) 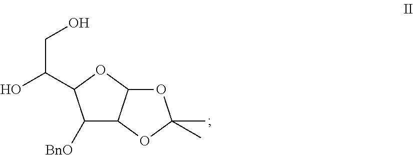 [SMILES: CC1(C)OC2OC(C(O)CO)C(OCc3ccccc3)C2O1]
c) subjecting the compound having the structure (II) to oxidation to obtain a compound (III) 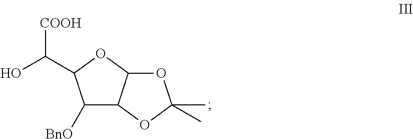 [SMILES: CC1(C)OC2OC(C(O)C(=O)O)C(OCc3ccccc3)C2O1]
d) subjecting the compound having the structure (III) to esterification to obtain a compound (IV) 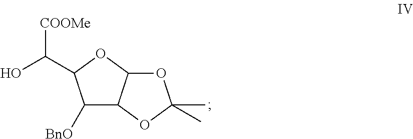 [SMILES: COC(=O)C(O)C1OC2OC(C)(C)OC2C1OCc1ccccc1]
e) treating the compound having the structure (IV) with triflic anhydride and pyridine in an organic solvent to obtain an O-protected compound having the structure (V) 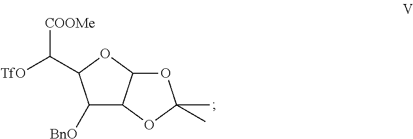 [SMILES: COC(=O)C(OS(=O)(=O)C(F)(F)F)C1OC2OC(C)(C)OC2C1OCc1ccccc1]
f) deprotecting and isomerization of the compound having the structure (V) to obtain a compound having the structure (VI) 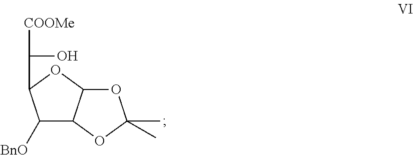 [SMILES: COC(=O)C(O)C1OC2OC(C)(C)OC2C1OCc1ccccc1]
g) deprotecting and performing ring expansion of the compound having the structure (VI) to obtain a compound having the structure (VII) 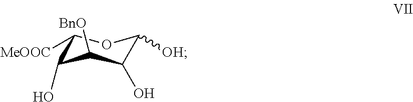
h) acetylating the compound having the structure (VII) to obtain a compound having the structure (VIII) 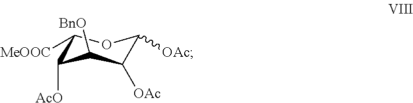
i) brominating the compound having the structure (VIII) to obtain a compound having the structure (IX) 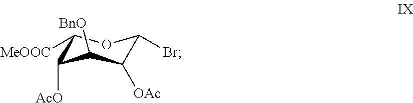
j) performing orthoesterification of the compound having the structure (IX) to obtain a compound having the structure (X) 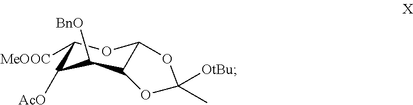
k) deacetylating the compound having the structure (X) to obtain a compound having the structure (XI); 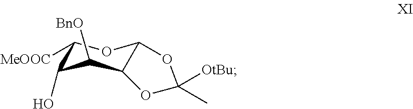
and
l) chloroacetylating the compound having the structure (XI) to obtain the compound having the structure (XII).

6. The process according to claim 1 wherein the step of subjecting the deacetylated pentasaccharide having the structure (XLIX) to O-sulfonation to provide an O-sulfonated pentasaccharide having the structure (L) 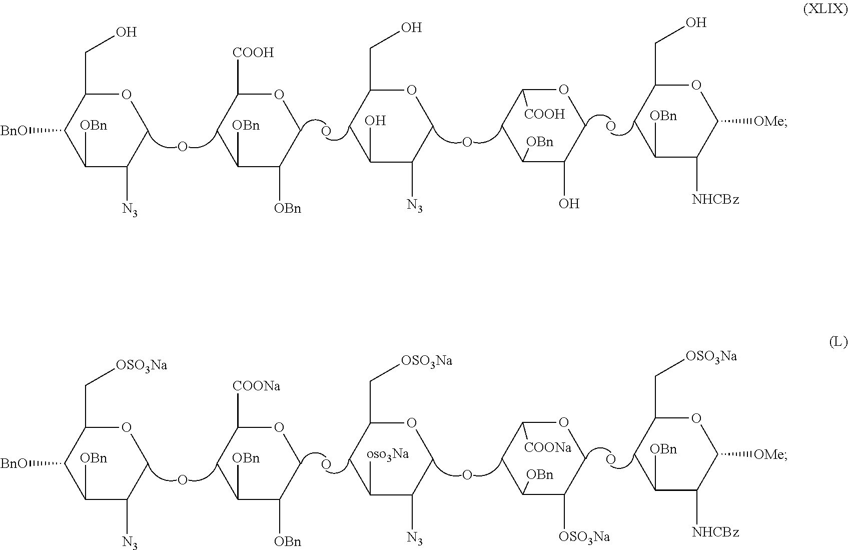
comprises subjecting the deacetylated pentasaccharide having the structure (XLIX) to O-sulfonation in DMF in the presence of SO₃-trimethylamine at 50° C. to 100° C. for 6 to 24 hours followed by removal of the DMF by distillation, salt removal and column purification, then pH adjustment.

7. A process for making a compound having the structure (XX) 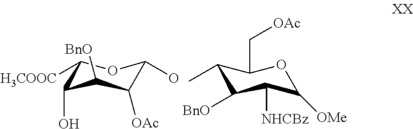
comprising linking the compound having the structure (XII) made in accordance with claim 5 with a compound having the structure (XVIII)

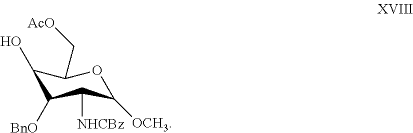

8. A process for making a monomer having the structure (XXVII) 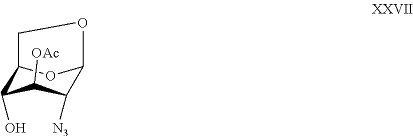 [SMILES: CC(=O)OC1[C@H](N=[N+]=[N-])C2OC[C@@H](O2)[C@H]1O]
comprising
a) acetylating D(+) glucose to produce a compound having the structure (XXI) 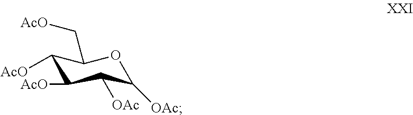
b) subjecting the compound having the structure (XXI) to treatment with HBr and zinc to produce a compound having the structure (XXII) 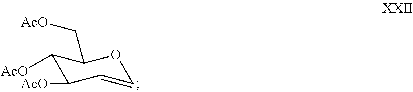 [SMILES: CC(=O)OCC1OC=CC(OC(C)=O)C1OC(C)=O]
c) combining the compound having the structure (XXII) with an organic solvent and subjecting the mixture to treatment with acetonitrile, bis (tis-n-butyl tin) oxide, and iodine to produce a compound having the structure (XXIII) 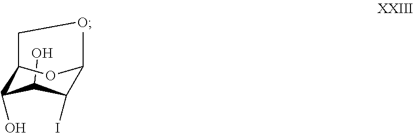 [SMILES: OC1[C@H](I)C2OC[C@@H](O2)[C@H]1O]
d) combining the compound having the structure (XXIII) with dimethylformamide, sodium bicarbonate in water and sodium azide to produce a compound having the structure (XXIV) 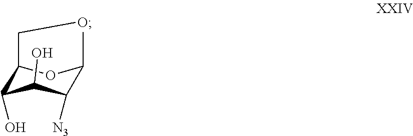 [SMILES: [N-]=[N+]=N[C@@H]1C2OC[C@@H](O2)[C@@H](O)C1O]
e) combining the compound having the structure (XXIV) with dichloromethane, imidazole and tert-butyldimethylsilyl ether (TBDMS) chloride to produce a compound having the structure (XXV) 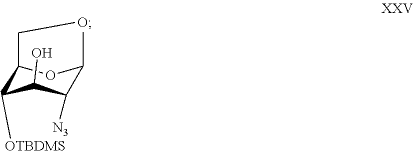 [SMILES: CC(C)(C)[Si](C)(C)O[C@H]1C(O)[C@H](N=[N+]=[N-])C2OC[C@H]1O2]
f) combining the compound having the structure (XXV) with pyridine and acetyl chloride to produce a compound having the structure (XXVI) 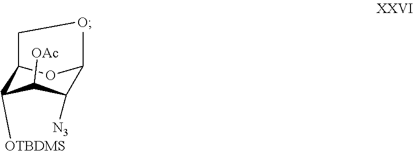 [SMILES: CC(=O)OC1[C@H](N=[N+]=[N-])C2OC[C@@H](O2)[C@H]1O[Si](C)(C)C(C)(C)C]
and
g) combining the compound having the structure (XXVI) with trifluoroacetic acid and subsequently adding dichloromethane, recovering crude product and purifying the product to obtain the compound having the structure (XXVII).

9. A process for making a compound having the structure (XLIII) 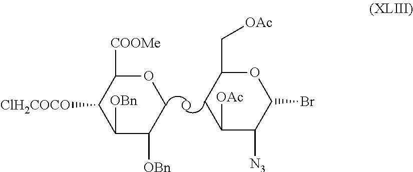
comprising combining the compound having the structure (XXVII) made in accordance with claim 8 with dichloromethane, adding molecular sieve to the reaction mass, adding mercuric bromide to the reaction mass, preparing a compound having the structure (XXXIX) 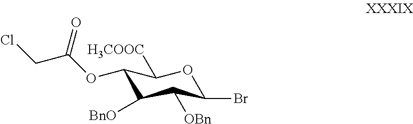
in dichloromethane, adding the compound (XXXIX) solution into the reaction mass, recovering a crude product, purifying the crude product using column chromatography, recovering a disaccharide having the structure (XL) 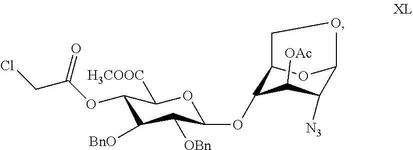
combining the disaccharide having the structure XL with acetic anhydride followed by addition of trifluoroacetic acid (TFA) and recovering a compound having the structure (XLII) 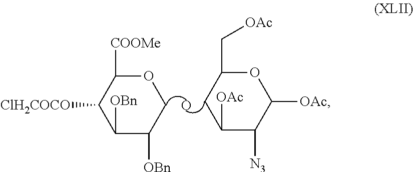
recrystallizing the compound having the structure (XLII),
adding dichloromethane to the compound having the structure (XLII), followed by addition of ethyl acetate, then adding titanium bromide, recovering the compound having the structure (XLIII) and recrystallizing it.

10. A process for making a compound having the structure (XLI) 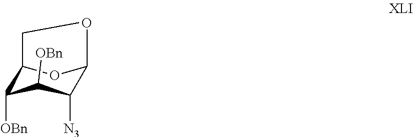 [SMILES: [N-]=[N+]=N[C@@H]1C2OC[C@@H](O2)[C@@H](OCc2ccccc2)C1OCc1ccccc1]
comprising combining a compound having the structure (XXIV) 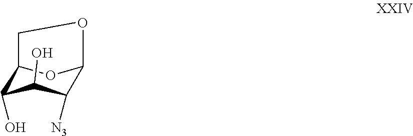 [SMILES: [N-]=[N+]=N[C@@H]1C2OC[C@@H](O2)[C@@H](O)C1O]
with toluene, N-methylpyrrolidone (NMP) and tetra butyl ammonium bromide (TBAB), subsequently adding benzyl chloride, and recovering the compound having the structure (XLI).

11. A process for making a compound having the structure (XLV) 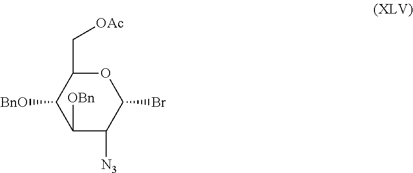 [SMILES: CC(=O)OCC1O[C@H](Br)C(N=[N+]=[N-])C(OCc2ccccc2)[C@@H]1OCc1ccccc1]
comprising acetylating a monosugar having the structure (XLI) at its anomeric carbon using AC₂O and trifluoroacetic acid (TFA) to produce a compound having the structure (XLIV) 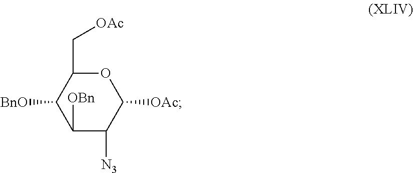 [SMILES: CC(=O)OCC1O[C@H](OC(C)=O)C(N=[N+]=[N-])C(OCc2ccccc2)[C@@H]1OCc1ccccc1]
recrystallizing the compound having the structure (XLIV),
brominating the compound having the structure (XLIV) at the anomeric carbon using titaniumtetrabromide in dichloromethane and ethyl acetate to yield the compound having the structure (XLV), and recrystallizing it in a solvent.

12. A process for making an O-sulfonated compound having the structure (L) 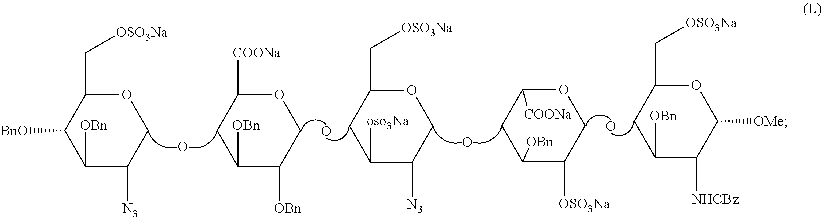
comprising providing a pentasaccharide having the structure (XLVIII) 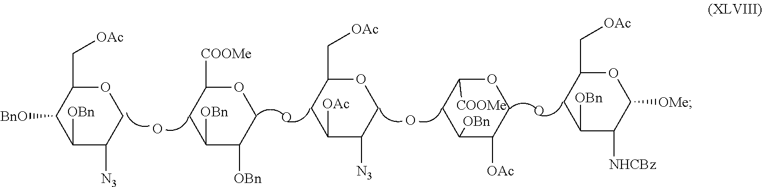
converting the pentasaccharide having the structure (XLVIII) by deacetylation to a deacetylated pentasaccharide having the structure (XLIX) 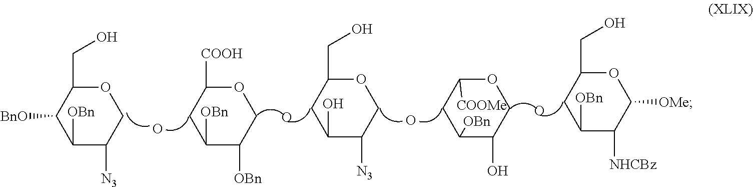
and
subjecting the deacetylated pentasaccharide having the structure (XLIX) to O-sulfonation in DMF in the presence of SO₃-trimethylamine at 50° C. to 100° C. for 6-24 hrs followed by removal of the solvent DMF by distillation, salt removal and column purification, then adjusting the pH to provide an O-sulfonated pentasaccharide having the structure (L).

13. The process according to claim 1, wherein the step of linking the dimers having the structures (XLIII) and (XX) to form a tetrasaccharide having the structure (XLVII) 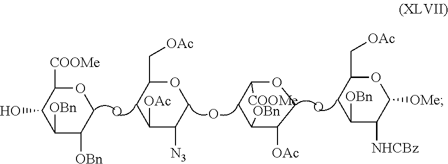
comprises the preparation of a tetrasaccharide having the structure (XLVI) 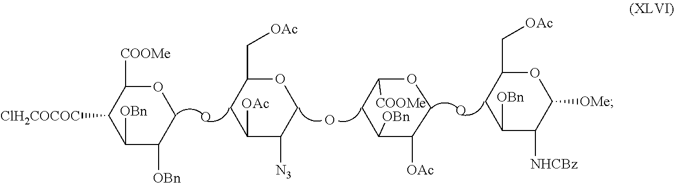
comprising combining the compound having the structure (XLIII) with a solvent and the compound having the structure (XX), reacting the mixture with silver carbonate and silver perchlorate anhydrous to create a reaction mass, diluting the reaction mass with a solvent, filtering the diluted reaction mass, obtaining a residue and purifying the reside with a methanol/water mixture to obtain the tetrasaccharide having the structure (XLVI).

14. The process according to claim 9 wherein the recrystallization of the compound having the structure (XLII) is performed using 10 volumes of diisopropyl ether.

15. The process according to claim 9 wherein the recrystallization of the compound having the structure (XLIII) is performed using 10 volumes of a mixture of isopropanol and methanol.

16. The process according to claim 11 wherein the recrystallization of the compound having the structure (XLIV) is performed using 6 volumes of diisopropyl ether.

17. The process according to claim 11 wherein the recrystallization of the compound having the structure (XLV) is performed using 10 volumes of diisopropyl ether.

18. The process according to claim 1 wherein the purification of fondaparinux free acid comprises treating the fondaparinux free acid in a mixture comprising acetone and water and treating the fondaparinux free acid in methanol.

19. The process according to claim 1 wherein the purification of fondaparinux sodium comprises treating the fondaparinux sodium in a mixture comprising methanol, water and sodium chloride.

20. A process for the preparation of fondaparinux sodium comprising:
a) providing monomers having the structures (XII), (XVIII), (XXVII), (XXXVIII) and (XLI) 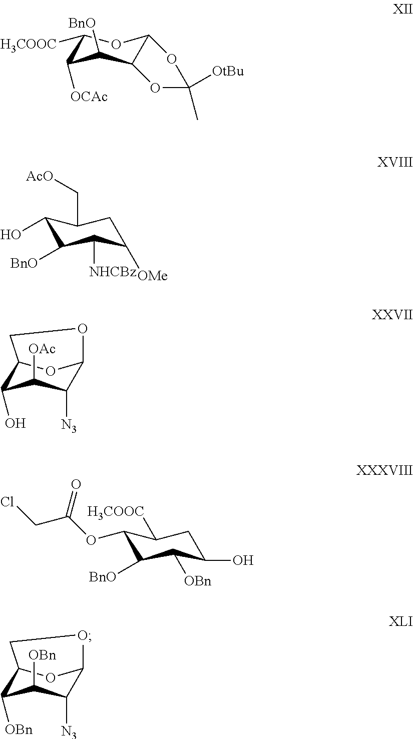
wherein the step of providing the monomer having the structure (XII) comprises
i) benzylating diacetone-d-glucose to obtain a benzylated diacetone d-glucose having the structure (I) 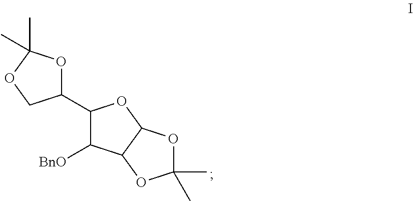
ii) deprotecting the benzylated diacetone-d-glucose having the structure (I) to obtain a compound having the structure (II) 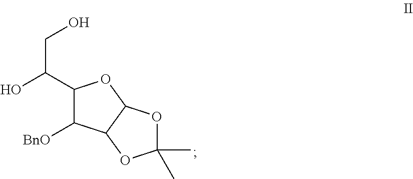 [SMILES: CC1(C)OC2OC(C(O)CO)C(OCc3ccccc3)C2O1]
iii) subjecting the compound having the structure (II) to oxidation to obtain a compound (III) 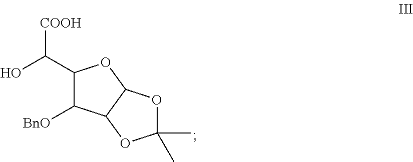 [SMILES: CC1(C)OC2OC(C(O)C(=O)O)C(OCc3ccccc3)C2O1]
iv) subjecting the compound having the structure (III) to esterification to obtain a compound (IV) 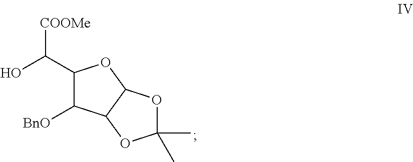 [SMILES: COC(=O)C(O)C1OC2OC(C)(C)OC2C1OCc1ccccc1]
v) treating the compound having the structure (IV) with triflic anhydride and pyridine in an organic solvent to obtain an O-protected compound having the structure (V) 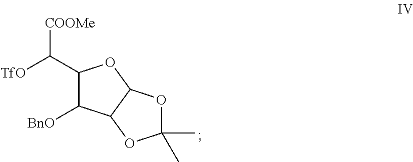 [SMILES: COC(=O)C(OS(=O)(=O)C(F)(F)F)C1OC2OC(C)(C)OC2C1OCc1ccccc1]
vi) deprotecting and isomerization of the compound having the structure (V) to obtain a compound having the structure (VI) 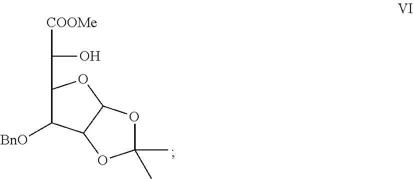 [SMILES: COC(=O)C(O)C1OC2OC(C)(C)OC2C1OCc1ccccc1]
vii) deprotecting and performing ring expansion of the compound having the structure (VI) to obtain a compound having the structure (VII) 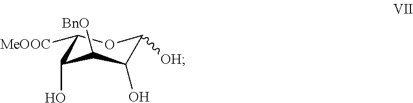
viii) acetylating the compound having the structure (VII) to obtain a compound having the structure (VIII) 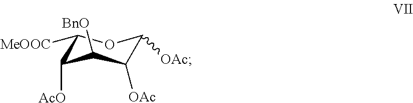
ix) brominating the compound having the structure (VIII) to obtain a compound having the structure (IX) 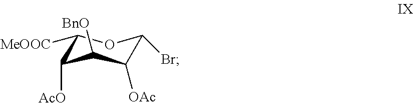
x) performing orthoesterification of the compound having the structure (IX) to obtain a compound having the structure (X) 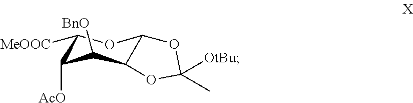
xi) deacetylating the compound having the structure (X) to obtain a compound having the structure (XI); 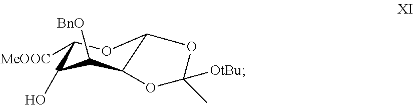
and
xii) chloroacetylating the compound having the structure (XI) to obtain the compound having the structure (XII);
b) linking the monomers having the structures (XII) and (XVIII) to form a disaccharide having the structure (XX) 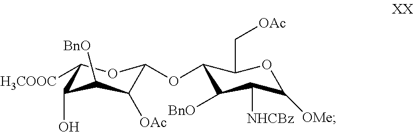
c) linking the monomers having the structures (XXXIX) and (XXVII) to form a disaccharide having the structure (XLIII) 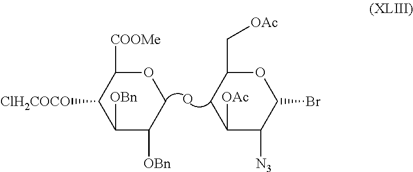
and purifying the disaccharide having the structure (XLIII) by recrystallization;
d) linking the dimers having the structures (XLIII) and (XX) to form a tetrasaccharide having the structure (XLVII) 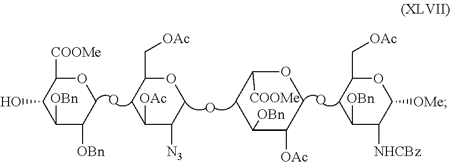
e) providing a monomer having the structure (XLV) 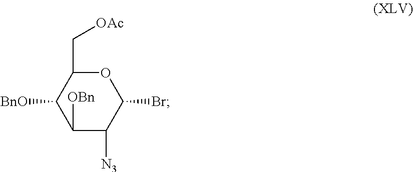 [SMILES: CC(=O)OCC1O[C@H](Br)C(N=[N+]=[N-])C(OCc2ccccc2)[C@@H]1OCc1ccccc1]
and purifying it by recrystallization;
f) linking the monomer having the structure (XLV) to the tetrasaccharide having the structure (XLVII) to form a pentasaccharide having the structure (XLVIII) 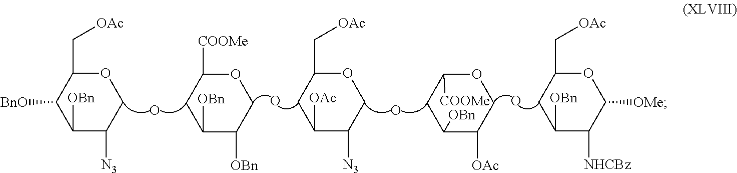
g) converting the pentasaccharide having the structure (XLVIII) by deacetylation to a deacetylated pentasaccharide having the structure (XLIX) 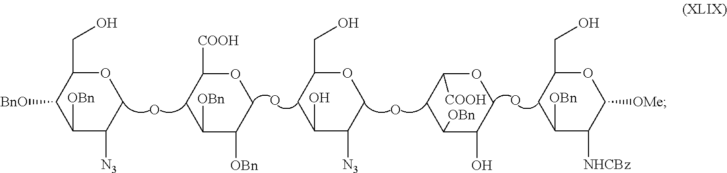
h) subjecting the deacetylated pentasaccharide having the structure (XLIX) to O-sulfonation in DMF in the presence of SO₃-trimethylamine followed by removing the solvent DMF by distillation, salt removal and column purification, then pH adjustment to provide an O-sulfonated pentasaccharide having the structure (L) 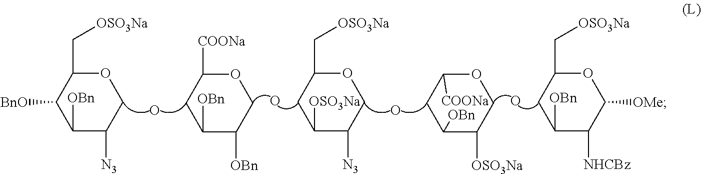
i) converting the O-sulfonated pentasaccharide having the structure (L) to a deprotected pentasaccharide having the structure (LI) 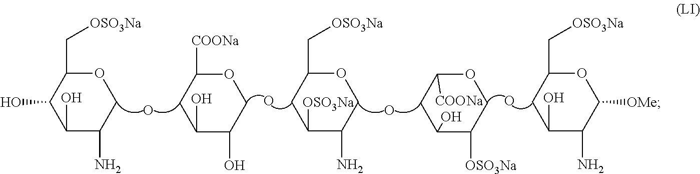
j) converting the deprotected pentasaccharide having the structure (LI) to fondaparinux free acid and purifying the fondaparinux free acid using at least one solvent; and
k) converting the fondaparinux free acid to fondaparinux sodium and purifying the fonadaparinux sodium.

21. A process for the preparation of fondaparinux sodium comprising:
a) providing monomers having the structures (XII), (XVIII), (XXVII), (XXXVIII) and (XLI) 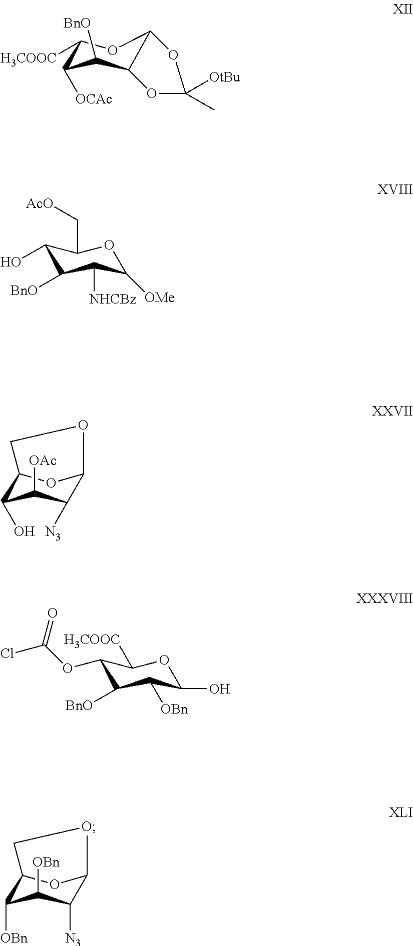
wherein the step of providing the monomer having the structure (XXVII) 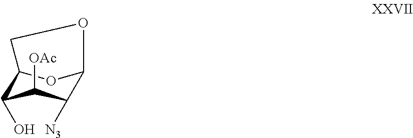 [SMILES: CC(=O)OC1[C@@H](N=[N+]=[N-])C2OC[C@@H](O2)[C@H]1O]
comprises
i) acetylating D(+) glucose to produce a compound having the structure (XXI) 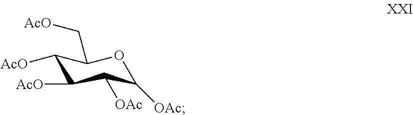
ii) subjecting the compound having the structure (XXI) to treatment with HBr and zinc to produce a compound having the structure (XXII) 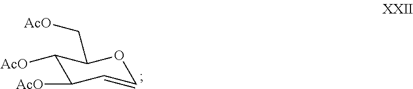 [SMILES: CC(=O)OCC1OC=CC(OC(C)=O)C1OC(C)=O]
iii) combining the compound having the structure (XXII) with an organic solvent and subjecting the mixture to treatment with acetonitrile, bis (tis-n-butyl tin) oxide, and iodine to produce a compound having the structure (XXIII) 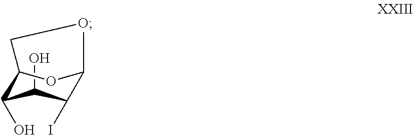 [SMILES: OC1[C@@H](I)C2OC[C@@H](O2)[C@H]1O]
iv) combining the compound having the structure (XIII) with dimethylformamide, sodium bicarbonate in water and sodium azide to produce a compound having the structure (XXIV) 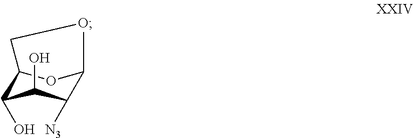 [SMILES: [N-]=[N+]=N[C@H]1C2OC[C@@H](O2)[C@@H](O)C1O]
v) combining the compound having the structure (XXIV) with dichloromethane, imidazole and tert-butyldimethylsilyl ether (TBDMS) chloride to produce a compound having the structure (XXV) 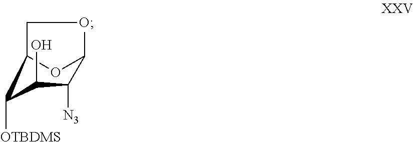 [SMILES: CC(C)(C)[Si](C)(C)O[C@H]1C(O)[C@H](N=[N+]=[N-])C2OC[C@H]1O2]
vi) combining the compound having the structure (XXV) with pyridine and acetyl chloride to produce a compound having the structure (XXVI) 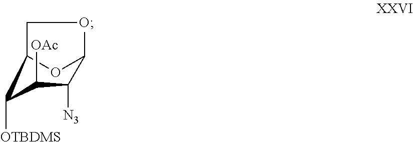 [SMILES: CC(=O)OC1[C@H](N=[N+]=[N-])C2OC[C@@H](O2)[C@H]1O[Si](C)(C)C(C)(C)C]
and
vii) combining the compound having the structure (XXVI) with trifluoroacetic acid and subsequently adding dichloromethane, recovering crude product and purifying the product to obtain the compound having the structure (XXVII);
b) linking the monomers having the structures (XII) and (XVIII) to form a disaccharide having the structure (XX) 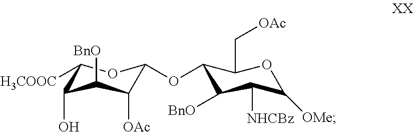
c) linking the monomers having the structures (XXXIX) and (XXVII) to form a disaccharide having the structure (XLIII) 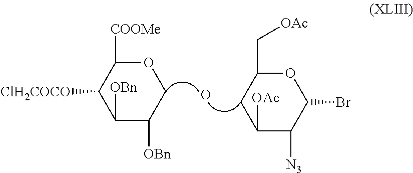
and purifying the disaccharide having the structure (XLIII) by recrystallization;
d) linking the dimers having the structures (XLIII) and (XX) to form a tetrasaccharide having the structure (XLVII) 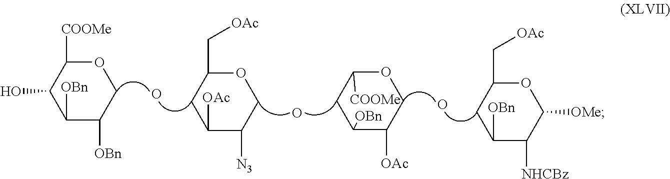
e) providing a monomer having the structure (XLV) 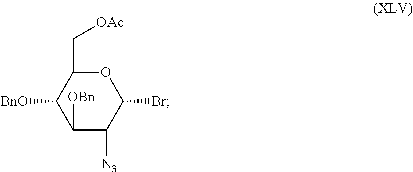 [SMILES: CC(=O)OCC1O[C@H](Br)C(N=[N+]=[N-])C(OCc2ccccc2)[C@@H]1OCc1ccccc1]
and purifying it by recrystallization;
f) linking the monomer having the structure (XLV) to the tetrasaccharide having the structure (XLVII) to form a pentasaccharide having the structure (XLVIII) 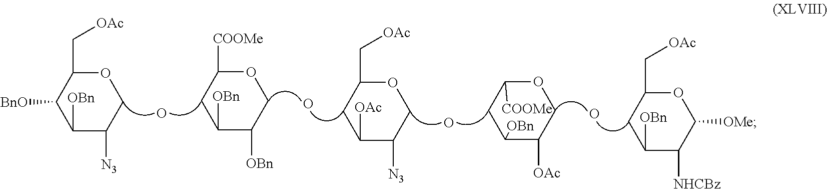
g) converting the pentasaccharide having the structure (XLVIII) by deacetylation to a deacetylated pentasaccharide having the structure (XLIX) 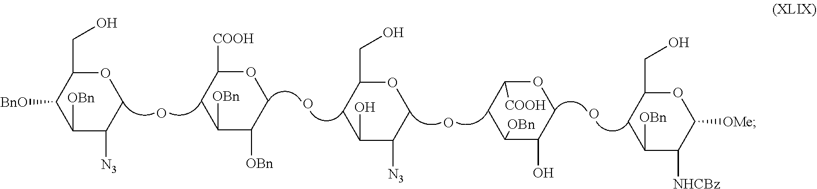
h) subjecting the deacetylated pentasaccharide having the structure (XLIX) to O-sulfonation in DMF in the presence of SO₃-trimethylamine followed by removing the solvent DMF by distillation, salt removal and column purification, then pH adjustment to provide an O-sulfonated pentasaccharide having the structure (L) 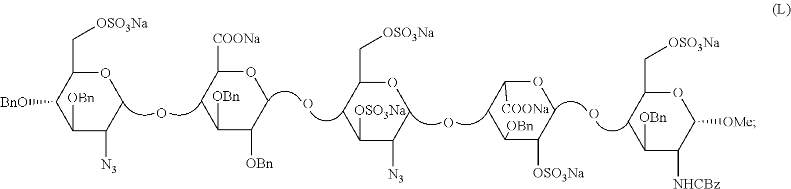
i) converting the O-sulfonated pentasaccharide having the structure (L) to a deprotected pentasaccharide having the structure (LI) 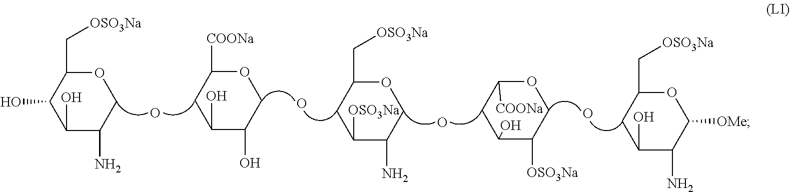
j) converting the deprotected pentasaccharide having the structure (LI) to fondaparinux free acid and purifying the fondaparinux free acid using at least one solvent; and
k) converting the fondaparinux free acid to fondaparinux sodium and purifying the fonadaparinux sodium.

22. A process for the preparation of fondaparinux sodium comprising:
a) providing monomers having the structures (XII), (XVIII), (XXVII), (XXXVIII) and (XLI) 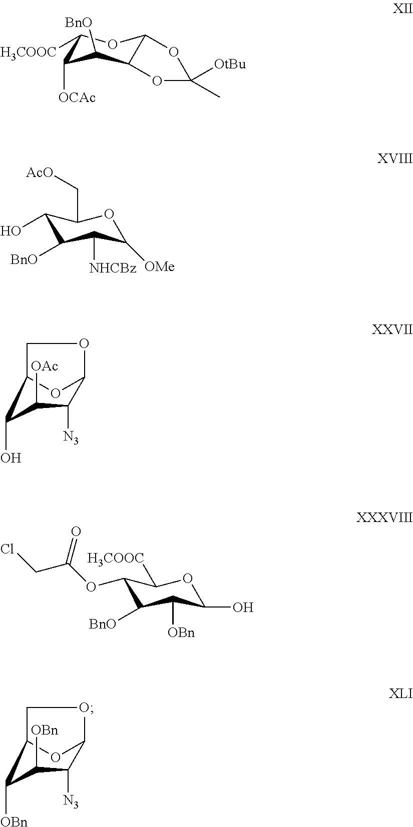
b) linking the monomers having the structures (XII) and (XVIII) to form a disaccharide having the structure (XX) 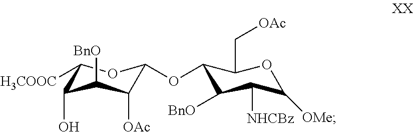
c) linking the monomers having the structures (XXXIX) and (XXVII) to form a disaccharide having the structure (XLIII) 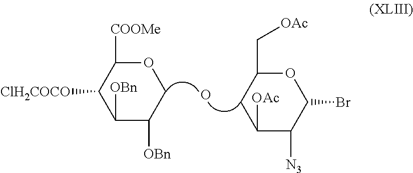
and purifying the disaccharide having the structure (XLIII) by recrystallization;
d) linking the dimers having the structures (XLIII) and (XX) to form a tetrasaccharide having the structure (XLVII) 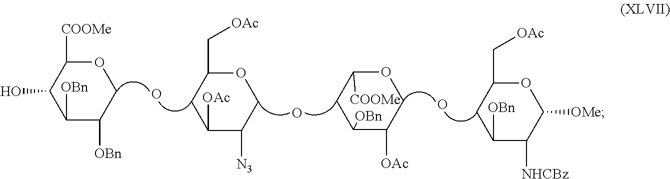
e) providing a monomer having the structure (XLV) 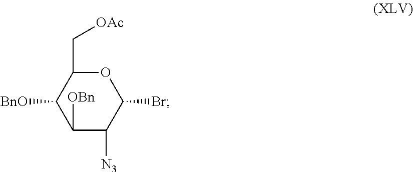 [SMILES: CC(=O)OCC1O[C@H](Br)C(N=[N+]=[N-])C(OCc2ccccc2)[C@@H]1OCc1ccccc1]
comprising the steps of
i) combining a compound having the structure (XXIV) with toluene, N-methylpyrrolidone (NMP) and tetra butyl ammonium bromide (TBAB), subsequently adding benzyl chloride, and recovering a monosugar having the structure (XLI) 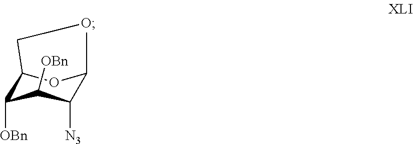 [SMILES: [N-]=[N+]=N[C@@H]1C2OC[C@@H](O2)[C@@H](OCc2ccccc2)C1OCc1ccccc1]
ii) acetylating the monosugar having the structure (XLI) at its anomeric carbon using AC₂O and trifluoroacetic acid (TFA) to produce a compound having the structure (XLIV) 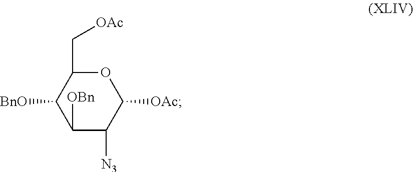 [SMILES: CC(=O)OCC1O[C@H](OC(C)=O)C(N=[N+]=[N-])C(OCc2ccccc2)[C@@H]1OCc1ccccc1]
and
iii) brominating the compound having the structure (XLIV) at the anomeric carbon using titanium tetrabromide in dichloromethane and ethyl acetate to yield the compound having the structure (XLV);
f) linking the monomer having the structure (XLV) to the tetrasaccharide having the structure (XLVII) to form a pentasaccharide having the structure (XLVIII) 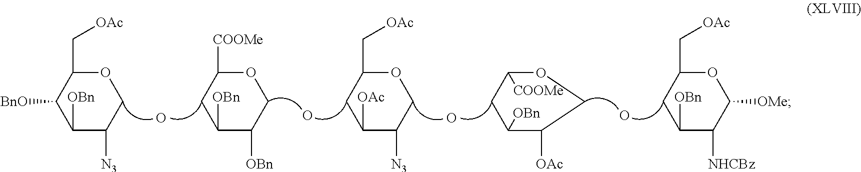
g) converting the pentasaccharide having the structure (XLVIII) by deacetylation to a deacetylated pentasaccharide having the structure (XLIX) 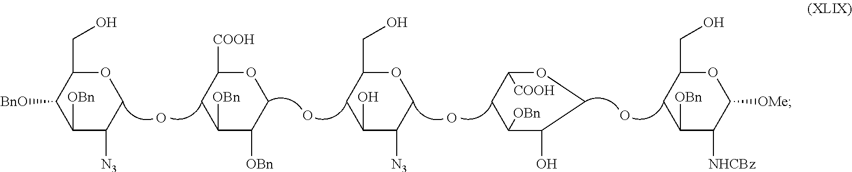
h) subjecting the deacetylated pentasaccharide having the structure (XLIX) to O-sulfonation in DMF in the presence of SO₃-trimethylamine followed by removing the solvent DMF by distillation, salt removal and column purification, then pH adjustment to provide an O-sulfonated pentasaccharide having the structure (L) 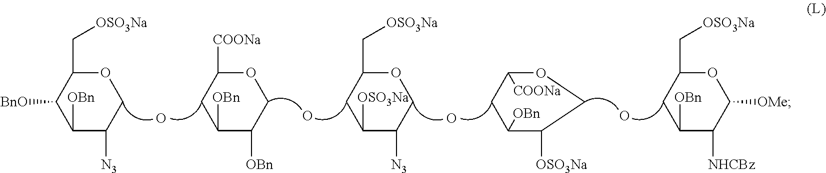
i) converting the O-sulfonated pentasaccharide having the structure (L) to a deprotected pentasaccharide having the structure (LI) 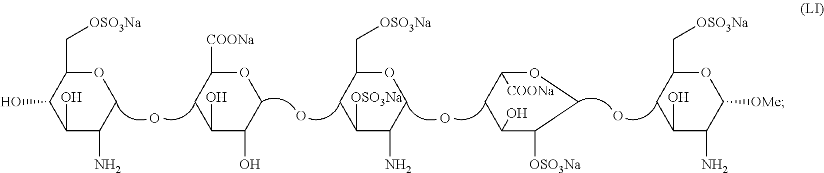
j) converting the deprotected pentasaccharide having the structure (LI) to fondaparinux free acid and purifying the fondaparinux free acid using at least one solvent; and
k) converting the fondaparinux free acid to fondaparinux sodium and purifying the fonadaparinux sodium.

23. A process for the preparation of fondaparinux sodium comprising:
a) providing monomers having the structures (XII), (XVIII), (XXVII), (XXXVIII) and (XLI) 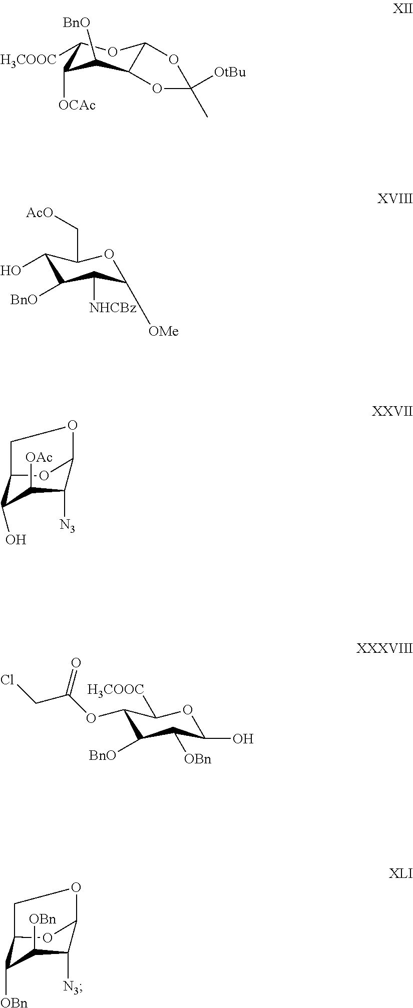
wherein the compound having the structure (XLI) is made by a process comprising combining a compound having the structure (XXIV) 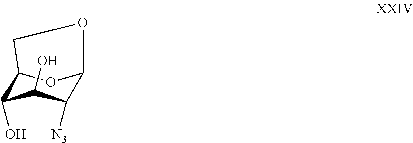 [SMILES: [N-]=[N+]=N[C@@H]1C2OC[C@@H](O2)[C@@H](O)C1O]
with toluene, N-methylpyrrolidone (NMP) and tetra butyl ammonium bromide (TBAB), subsequently adding benzyl chloride, and recovering the compound having the structure (XLI);
b) linking the monomers having the structures (XII) and (XVIII) to form a disaccharide having the structure (XX) 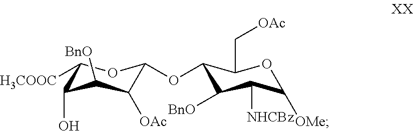
c) linking the monomers having the structures (XXXIX) and (XXVII) to form a disaccharide having the structure (XLIII) 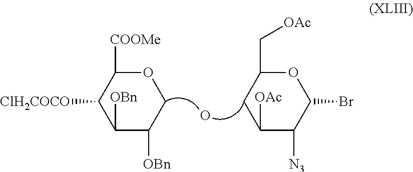
and purifying the disaccharide having the structure (XLIII) by recrystallization;
d) linking the dimers having the structures (XLIII) and (XX) to form a tetrasaccharide having the structure (XLVII) 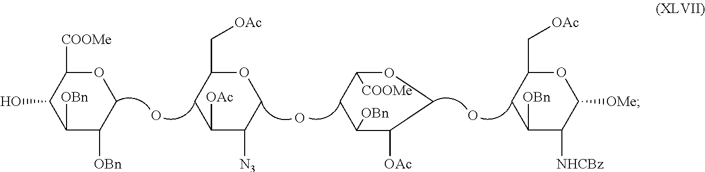
e) providing a monomer having the structure (XLV) 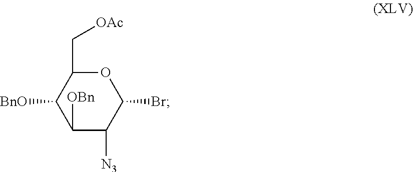 [SMILES: CC(=O)OCC1O[C@H](Br)C(N=[N+]=[N-])C(OCc2ccccc2)[C@@H]1OCc1ccccc1]
and purifying it by recrystallization;
f) linking the monomer having the structure (XLV) to the tetrasaccharide having the structure (XLVII) to form a pentasaccharide having the structure (XLVIII) 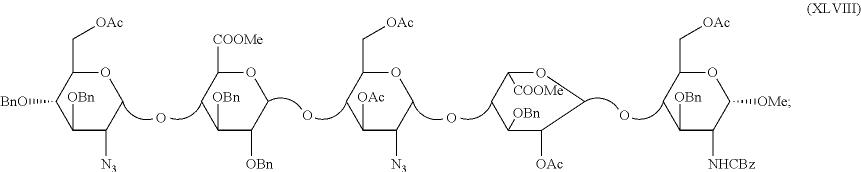
g) converting the pentasaccharide having the structure (XLVIII) by deacetylation to a deacetylated pentasaccharide having the structure (XLIX) 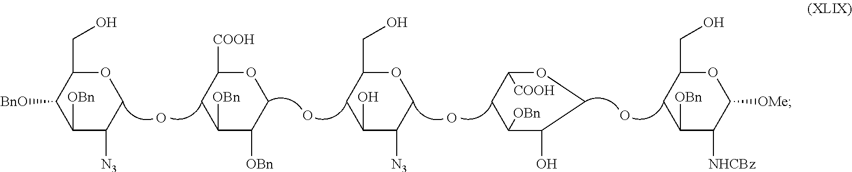
h) subjecting the deacetylated pentasaccharide having the structure (XLIX) to O-sulfonation in DMF in the presence of SO₃-trimethylamine followed by removing the solvent DMF by distillation, salt removal and column purification, then pH adjustment to provide an O-sulfonated pentasaccharide having the structure (L) 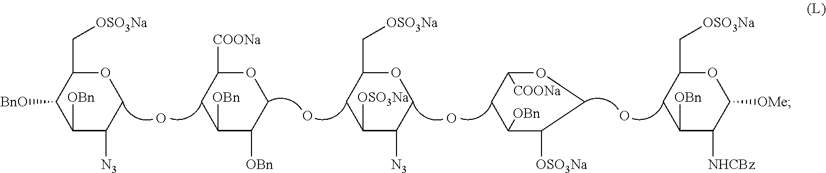
i) converting the O-sulfonated pentasaccharide having the structure (L) to a deprotected pentasaccharide having the structure (LI) 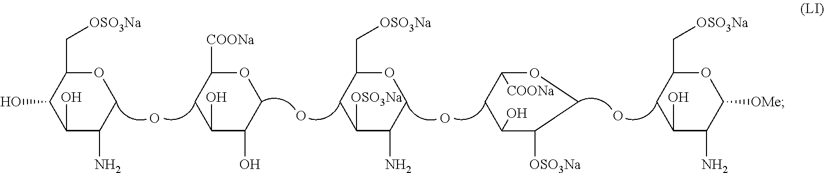
j) converting the deprotected pentasaccharide having the structure (LI) to fondaparinux free acid and purifying the fondaparinux free acid using at least one solvent; and
k) converting the fondaparinux free acid to fondaparinux sodium and purifying the fonadaparinux sodium.

24. A process for the preparation of pharmaceutical grade fondaparinux sodium having a purity of greater than 99.8% by HPLC comprising the method according to claim 1.

25. Pharmaceutical grade fondaparinux sodium having a purity of greater than 99.8% by HPLC made according to the method of claim 1.

26. Fondaparinux sodium having a purity of greater than 99.8% by HPLC.
